(19) United States
(12) Patent Application Publication
(10) Pub. No.: US 2022/0125938 A1
(43) Pub. Date: Apr. 28, 2022

(54) COMPOSITIONS AND METHODS FOR DISASSEMBLING AMYLOID FIBRILS

(71) Applicant: The Board of Trustees of the University of Illinois, Urbana, IL (US)

(72) Inventors: Edwin G. MOORE, Round Lake, IL (US); Jeffrey S. MOORE, Savoy, IL (US); Yang SONG, Framingham, MA (US)

(73) Assignee: The Board of Trustees of the University of Illinois, Urbana, IL (US)

(21) Appl. No.: 17/567,667

(22) Filed: Jan. 3, 2022

Related U.S. Application Data
(62) Division of application No. 15/943,433, filed on Apr. 2, 2018.
(60) Provisional application No. 62/486,103, filed on Apr. 17, 2017.

(51) Int. Cl. A61K 47/59; A61P 25/28; C07K 17/08; C07K 14/47

(52) U.S. Cl. CPC A61K 47/595; A61P 25/28; A61K 47/59; C07K 14/4711; C07K 17/08

(57) ABSTRACT

The present disclosure provides multivalent polymer-peptide conjugate compositions capable of breaking already formed amyloid fibrils. Also provided are methods of treating a subject having or suspected of having Alzheimer's disease by administering a therapeutically effective amount of these multivalent polymer-peptide conjugate compositions.

### RELATED APPLICATIONS

This application claims priority under 35 U.S.C. § 119(e) to U.S. Provisional Patent Application No. 62/486,103, filed Apr. 17, 2017, which is incorporated herein by reference.

### GOVERNMENT SUPPORT

This invention was made with government support under Grant No. DE-FG02-02ER46471 awarded by the Department of Energy. The government has certain rights in the invention.

### BACKGROUND OF THE INVENTION

Protein misfolding and aggregation are molecular self-assembly processes that are associated with lethal human diseases, including Alzheimer's disease, Parkinson's disease, and type II diabetes. The hallmark of most of these diseases is the formation of highly ordered and β-sheet rich aggregates known as amyloid fibrils. Inhibitory modulation is a common strategy to prevent amyloid fibril formation and thus reduce cytotoxicity of amyloid aggregates. Many inhibitory modulators have been developed and shown therapeutic potency, including small molecule-, peptide-, antibody-, and nanomaterial-based inhibitors. Compared to the widely-studied modulation and prevention of amyloid aggregation by synthetic agents, disassembly of preformed amyloid fibrils remains largely unknown and challenging. Very few molecules have been reported to break down amyloid fibrils, since amyloid fibrils are hypothesized to be the lowest free energy state among all aggregated species and monomer.

We previously reported the design and synthesis of multivalent polymer-iAβ₅ conjugates (mP-iAβ₅) in which LPFFD (iAβ₅) was selected as the peptide moiety for mPPCs, we also previously demonstrated the inhibitory effect of mP-iAβ₅ conjugates on Aβ₄₀ fibrillation by modulating the nucleation kinetics for Aβ₄₀ fibril formation. Peptide iAβ₅ (49 kDa) binds to the central hydrophobic sequence Aβ₁₇₋₂₁ (LVFFA) of Aβ₄₀ with specificity to interfere with the β-sheet interactions between interstrand LVFFA motifs. Through specific peptide interactions and multivalent effect, mP-iAβ₅ conjugates stabilized transient intermediates of Aβ₄₀ oligomerization into discrete nanostructures. The previous work has demonstrated that mP-iAβ₅ conjugates having an average of 7 mol % iAβ₅ peptide per polymer chain achieved the optimum inhibitory effect.

Although an improved inhibitory effect can be achieved, for example, with an optimal mol % of an Aβ peptide per polymer chain, other drug-like properties are needed to achieve efficacy in reducing or preventing amyloid fibril formation.

### SUMMARY

Disclosed herein is the discovery that multivalent polymers capable of disassembling amyloid fibrils show improved efficacy at higher molecular weights. Accordingly, this disclosure provides a multivalent random copolymer comprising Formula I:

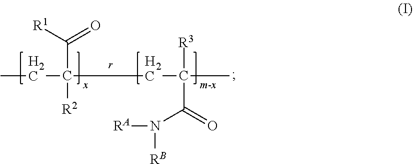

wherein

R¹ is an Amyloid β binding peptide;

R² and R³ are each independently (C₁-C₃)alkyl;

R^{A} is H or methyl;

R^{B} is (C₁-C₆)alkyl or (C₃-C₆)cycloalkyl wherein the alkyl or cycloalkyl is optionally monosubstituted with OH or NH₂;

m is 100 to 2000;

x is 1 to 200; and

the number average molecular weight of the copolymer is about 20 kDa to about 500 kDa;

and wherein the r between the x and m-x segments indicates that the copolymer is a random copolymer.

Additionally, this disclosure provides a method of disassembling an amyloid fibril comprising contacting an amyloid fibril with a multivalent copolymer of Formula I, wherein the copolymer binds to the amyloid fibril and at least partially disassembles the secondary structure of the amyloid fibril into one or more nanostructures having a length of less than about 400 nm.

Also, this disclosure provides a method of inhibiting the proliferation of amyloid fibrils in a subject at risk of developing amyloid β plaques comprising administering to the subject a multivalent copolymer of Formula I, wherein the copolymer binds to an amyloid oligomer in the subject, thereby inhibiting a proliferation of amyloid fibrils and formation of amyloid β plaques.

The disclosure also provides novel multivalent copolymers of Formula I, intermediates for the synthesis of copolymers of Formula I, as well as methods of preparing copolymers of Formula I. The disclosure additionally provides monomers of Formula I that are useful as intermediates for the synthesis of other useful monomer and compounds. The disclosure provides for the use of copolymers of Formula I and for the manufacture of copolymers of Formula I.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Design and structure of mP-iAβ₅ multivalent polymer—peptide conjugates. PHPMA stands for poly(hydroxypropyl methacrylamide). The average number (x) of peptide moieties per chain and the number-average degree of polymerization (m) are shown for each mP-iAβ₅ conjugate.

FIG. 2. Disassembly effects on preincubated Aβ₄₀ (15 μM in 10 mM PBS buffer) fibrils by 1.0 equiv mP-iAβ₅ conjugates of different molecular weight. AFM (top) and DLS (bottom) were recorded after 3 days. The scale bars for AFM images are 500 nm.

FIG. 3. Disassembly effects on preincubated Aβ₄₀ (15 μM in 10 mM PBS buffer) fibrils by mP-iAβ₅ conjugates of different molecular weight at a fixed total concentration of iAβ₅ moieties. AFM (top) and DLS (bottom) were recorded after 3 days. The scale bars for AFM images are 500 nm.

FIG. 4. Postulated pathways on Aβ₄₀ fibril disassembly by mP-iAβ₅ conjugates: (a) mP-iAβ₅ conjugates interact with Aβ₄₀ fibrils and disassemble Aβ₄₀ fibrils into β-structured complex, which is congruent with CD and ThT results. (b) Aβ₄₀ fibrils are in equilibrium with monomeric/oligomeric Aβ₄₀ peptide, and mP-iAβ₅ conjugates interact with monomeric/oligomeric Aβ₄₀ peptide, which shift the equilibrium to the disassembly direction. The result is a random coil complex.

FIG. 5. Reaction coordinate of Aβ₄₀ aggregation pathway without mP-iAβ₅ conjugates (solid lines), and the two roles that mP-iAβ₅ conjugates exhibit on Aβ₄₀ aggregation (dashed lines). Inhibition of fibrillation occurs when Aβ₄₀ monomer is mixed with mP-iAβ₅ conjugates, resulting in a random coil complex (dashed lines from the left). Aβ₄₀ fibril disassembly occurs when mature Aβ₄₀ fibrils are mixed with mP-iAβ₅ conjugates, resulting in a β-structured complex (dashed lines from the right). Aβ₄₀/mP-iAβ₅ complex generated from Aβ₄₀ fibrils and that generated from Aβ₄₀ monomer do not interconvert.

FIG. 6. Synthesis of mP-iAβ₅ conjugates 4 and control polymer 5 (Scheme 1).

FIG. 7. GPC trace of mP-iAβ₅ conjugates 4.

FIG. 8. The aggregation of Aβ₄₀ control monitored by ThT assays.

FIG. 9. Effects of 90 kDa mP-iAβ₅ conjugates (1.0 equiv), PHPMA (1.0 equiv), and iAβ₅ (32.5 equiv) on Aβ₄₀ disassembly monitored by ThT assays. We define equiv as the molar ratio of mP-iAβ₅ conjugates or iAβ₅ peptide to Aβ₄₀. ThT assays were performed on 15 μM Aβ₄₀ peptide in PBS buffer (pH 7.4) at 37° C. with shaking (567 rpm).

FIG. 10. Effects of 90 kDa mP-iAβ₅ conjugates (0.5 equiv), PHPMA (0.5 equiv), and iAβ₅ (16.7 equiv) on Aβ₄₀ disassembly monitored by ThT assays. We define equiv as the molar ratio of mP-iAβ₅ conjugates or iAβ₅ peptide to Aβ₄₀. ThT assays were performed on 15 μM Aβ₄₀ peptide in PBS buffer (pH 7.4) at 37° C. with shaking (567 rpm).

FIG. 11. Disassembly effects on pre-incubated Aβ₄₀ (15 μM in 10 mM PBS buffer) fibrils by 1.0 equiv of 90 kDa mP-iAβ₅ conjugates. AFM (top) and DLS (bottom) were recorded over 1 day (a, a′), 2 days (b,b′), and 3 days (c, c′). We define equiv as molar ratio of mP-iAβ₅ conjugates to Aβ₄₀.

FIG. 12. Disassembly effects on pre-incubated Aβ₄₀ (15 μM in 10 mM PBS buffer) fibrils by 0.5 equiv of 90 kDa mP-iAβ₅ conjugates. AFM (top) and DLS (bottom) were recorded over 1 day (a, a′), 2 days (b,b′), and 3 days (c, c′). We define equiv as molar ratio of mP-iAβ₅ conjugates to Aβ₄₀.

FIG. 13. Disassembly effects on pre-incubated Aβ₄₀ (15 μM in 10 mM PBS buffer) fibrils by 0.2 equiv of 224 kDa mP-iAβ₅ conjugates. AFM (top) and DLS (bottom) were recorded over 1 day (a, a′), 2 days (b,b′), and 3 days (c, c′). We define equiv as molar ratio of mP-iAβ₅ conjugates to Aβ₄₀.

FIG. 14. CD spectra. Secondary structures of Aβ₄₀ (15 μM) fibrils without fibril breakers (a), and with 15 μM of 166 kDa mP-iAβ₅ conjugates, 1.0 equiv (b), 910 μM of iAβ₅, 60.7 equiv (c), and 15 μM of 166 kDa PHPMA, 1.0 equiv (d). CD measurements were in a 10 mM sodium phosphate buffer (pH 7.4) with continuous shaking (567 rpm) at 37° C. We define equiv as the molar ratio of mP-iAβ₅ conjugates, iAβ₅, and PHPMA to Aβ₄₀.

FIG. 15. CD spectra. Secondary structures of freshly prepared Aβ₄₀ (15 μM)(a), and with 15 μM of 166 kDa mP-iAβ₅ conjugates, 1.0 equiv (b), and 910 μM of iAβ₅, 60.7 equiv (c). CD measurements were in a 10 mM sodium phosphate buffer (pH 7.4) with continuous shaking (567 rpm) at 37° C. We define equiv as the molar ratio of mP-iAβ₅ conjugates, iAβ₅, and PHPMA to Aβ₄₀.

FIG. 16. Linear regression of ln[A]˜t when the total concentration of iAβ₅ moieties is kept constant.

FIG. 17. Quadratic regression of S_{Mn}˜Mn when the total concentration of iAβ₅ moieties is kept constant.

FIG. 18. Linear regression of ln[A]˜t when the mol concentration of mP-iAβ₅ is kept constant.

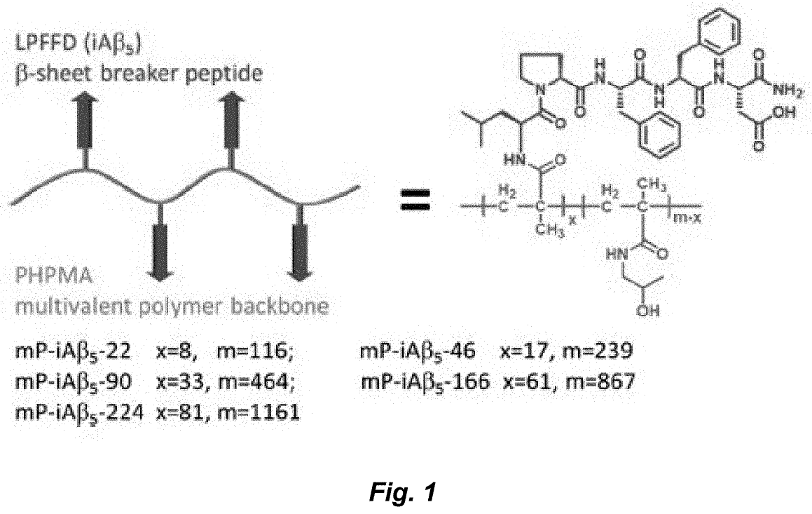

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the embodiments above and the claims below. Reference should therefore be made to the embodiments above and claims below for interpreting the scope of the invention.

### DETAILED DESCRIPTION

The compositions and methods now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements.

Likewise, many modifications and other embodiments of the compositions and methods described herein will come to mind to one of skill in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%. For example, repeat unit A is substantially soluble (e.g., greater than about 95% or greater than about 99%) in a polar organic solvent and is substantially insoluble (e.g., less than about 5% or less than about 1%) in a fluorocarbon solvent. In another example, repeat unit B is substantially soluble (e.g., greater than about 95% or greater than about 99%) in a fluorocarbon solvent and is substantially insoluble (e.g., less than about 5% or less than about 1%) in a polar organic solvent.

A “solvent” as described herein can include water or an organic solvent. Examples of organic solvents include hydrocarbons such as toluene, xylene, hexane, and heptane; chlorinated solvents such as methylene chloride, chloroform, and dichloroethane; ethers such as diethyl ether, tetrahydrofuran, and dibutyl ether; ketones such as acetone and 2-butanone; esters such as ethyl acetate and butyl acetate; nitriles such as acetonitrile; alcohols such as methanol, ethanol, and tert-butanol; and aprotic polar solvents such as N,N-dimethylformamide (DMF), N,N-dimethylacetamide (DMA), and dimethyl sulfoxide (DMSO). Solvents may be used alone or two or more of them may be mixed for use to provide a “solvent system”.

As used herein, the term “substituted” or “substituent” is intended to indicate that one or more (for example., 1-20 in various embodiments, 1-10 in other embodiments, 1, 2, 3, 4, or 5; in some embodiments 1, 2, or 3; and in other embodiments 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Additionally, the suitable indicated groups can include, e.g., —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, NC(═O)R, —C(═O)R, —C(═O)NRR—S(═O)₂O⁻, —S(═O)₂OH, —S(═O)₂R, —OS(═O)₂OR, —S(═O)₂NR, —S(═O)R, —OP(═O)O₂RR, —P(═O)O₂RR—P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, —C(NR)NRR, where each X is independently a halogen (“halo”): F, Cl, Br, or I; and each R is independently H, alkyl, aryl, heterocycle, protecting group or prodrug moiety. As would be readily understood by one skilled in the art, when a substituent is keto (i.e., ═O) or thioxo (i.e., ═S), or the like, then two hydrogen atoms on the substituted atom are replaced.

The term “alkyl” refers to a branched or unbranched hydrocarbon having, for example, from 1-20 carbon atoms, and often 1-12, 1-10, 1-8, 1-6, or 1-4 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below. The alkyl can also be optionally partially or fully unsaturated. As such, the recitation of an alkyl group can include both alkenyl and alkynyl groups. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3 -enyl, cyclohexyl, 1-cyclohex-1-enyl, 1-cyclohex-2-enyl, 1-cyclohex-3-enyl, and the like.

The term “aromatic” refers to either an aryl or heteroaryl group or substituent described herein. Additionally, an aromatic moiety may be a bisaromatic moiety, a trisaromatic moiety, and so on. A bisaromatic moiety has a single bond between two aromatic moieties such as, but not limited to, biphenyl, or bipyridine. Similarly, a trisaromatic moiety has a single bond between each aromatic moiety.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. In other embodiments, the aryl group can have 6 to 60 carbons atoms, 6 to 120 carbon atoms, or 6 to 240 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms. Examples of heteroaryl groups include, but are not limited to, 2H-pyrrolyl, 3H-indolyl, 4H-quinolizinyl, acridinyl, benzo[b]thienyl, benzothiazolyl, P-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

The phrase “one or more” is readily understood by one of skill in the art, particularly when read in context of its usage. For example, one or more substituents on a phenyl ring refers to one to five, or one to up to four, for example if the phenyl ring is disubstituted. One or more subunits (i.e., repeat units or blocks) of a polymer can refer to about 5 to about 100,000, or any number of subunits.

Substituents of the compounds and polymers described herein may be present to a recursive degree. In this context, “recursive substituent” means that a substituent may recite another instance of itself. Because of the recursive nature of such substituents, theoretically, a large number may be present in any given claim. One of ordinary skill in the art of organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. Recursive substituents are an intended aspect of the invention. One of ordinary skill in the art of organic chemistry understands the versatility of such substituents. To the degree that recursive substituents are present in a claim of the invention, the total number in the repeating unit of a polymer example can be, for example, about 1-50, about 1-40, about 1-30, about 1-20, about 1-10, or about 1-5.

The term, “repeat unit”, “repeating unit”, or “block” as used herein refers to the moiety of a polymer that is repetitive. The repeat unit may comprise one or more repeat units, labeled as, for example, repeat unit A, repeat unit B, repeat unit C, etc. Repeat units A-C, for example, may be covalently bound together to form a combined repeat unit. Monomers or a combination of one or more different monomers can be combined to form a (combined) repeat unit of a polymer or copolymer.

The term “molecular weight” for the copolymers disclosed herein refers to the average number molecular weight (Mn). The corresponding weight average molecular weight (Mw) can be determined from other disclosed parameters by methods (e.g., by calculation) known to the skilled artisan.

### Embodiments of the Invention

This disclosure describes various embodiments of a multivalent random copolymer comprising Formula I:

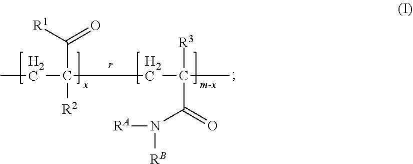

wherein

R¹ is an Amyloid β binding peptide;

R² and R³ are each independently (C₁-C₃)alkyl;

R^{A} is H or methyl;

R^{B} is (C₁-C₆)alkyl or (C₃-C₆)cycloalkyl wherein the alkyl or cycloalkyl is optionally monosubstituted with OH or NH₂;

m is 100 to 2000;

x is 1 to 200; and

the number average molecular weight of the copolymer is about 20 kDa to about 500 kDa;

and wherein the r between the x and m-x segments indicates that the copolymer is a random copolymer.

The multivalent copolymers disclosed herein can comprise random or block copolymers. However, the multivalent copolymers of Formula I described herein is random copolymer, as shown by the “r” over the bond between the x and m-x units of the multivalent copolymer (as would be readily recognized by the method of preparation of the multivalent copolymers as described, for example, in Example 4). Thus, the arrangement of the x units and m-x units is random throughout the length of the multivalent copolymer of the Formula I, and the total number of x units and m-x units is defined by x and m of Formula I, randomly arranged along the length of the multivalent copolymer.

In various embodiments, the ends of the copolymer (i.e., the initiator end or terminal end), is a low molecular weight moiety (e.g. under 500 Da), such as, H, OH, OOH, CH₂OH, CN, NH₂, or a hydrocarbon such as an alkyl (for example, a butyl or 2-cyanoprop-2-yl moiety at the initiator and terminal end), alkene or alkyne, or a moiety as a result of an elimination reaction at the first and/or last repeat unit in the copolymer (e.g., substituent Q of Scheme A).

In additional embodiments, R¹ is LPFFD (SEQ ID NO:1), LVFFA (SEQ ID NO:2), KLVFFA (SEQ ID NO:3), KLVFFAE (SEQ ID NO:4), AIIGL (SEQ ID NO:5), or AH(Met)GL (SEQ ID NO:6). In other embodiments, the multivalent copolymer may comprise more than one different R¹ group. In other embodiments, R¹ is LPFFD (SEQ ID NO:1), R¹ and R² are CH₃, R^{A} is H, and R^{B} is —CH₂CH(OH)CH₃.

In some embodiments, the binding peptide (R¹) has a loading ratio of about 1% to about 25% of the monomer segments of the copolymer. In other embodiments, the loading ratio is about 5% to about 10%. In yet other embodiments the loading ratio % is about 0.5%, about 1%, about 2%, about 3%, about 4%, about 5%, about 6%, about 7%, about 8%, about 9%, about 10%, about 11%, about 12%, about 13%, about 14%, or about 15%.

In other embodiments, m is about 100 to about 1200, noting that the copolymer is a random copolymer and the x units and m-x units of the formulas are randomly dispersed and not blocks. In other embodiments, m is about 100 to about 200, about 200 to about 900, or about 250 to about 750. In yet other embodiments, x is about 5 to about 75. In some embodiments, x is about 2 to about 15, about 15 to about 40, about 40 to about 60, or about 60 to about 80.

In various embodiments of the disclosure, the number average molecular weight of the copolymer is about 50 kDa to about 500 kDa. In various other embodiments, the number average molecular weight of the copolymer is about 50 kDa to about 300 kDa. In additional embodiments, the number average molecular weight of the copolymer is about 100 kDa to about 400 kDa. In yet other various embodiments, the number average molecular weight is about 100 kDa, about 125 kDa, about 150 kDa, about 175 kDa, about 200 kDa, about 225 kDa, about 250 kDa, about 275 kDa, about 300 kDa, about 325 kDa, about 350 kDa, about 375 kDa, about 400 kDa, about 425 kDa, about 450, about 475 kDa, about 500 kDa, about 550 kDa, about 600 kDa, about 700 kDa, about 800 kDa, about 900 kDa, about 1250 kDa, or about 1500 kDa.

This disclosure also provides a method of disassembling an amyloid fibril comprising contacting an amyloid fibril with a multivalent copolymer disclosed herein, wherein the copolymer binds to the amyloid fibril and at least partially disassembles the secondary structure of the amyloid fibril into one or more nanostructures having a length of less than about 400 nm.

In some embodiments, the nanostructures have a length of less than 100 nm. In other embodiments, the nanostructures have a diameter of less than 100 nm. In additional embodiments, the copolymer has a number average molecular weight of about 100 kDa to about 400 kDa and R¹ is LPFFD (SEQ ID NO:1). In yet other embodiments, the nanostructures are less hydrophobic than the amyloid fibril.

In various embodiments, disassembling a plurality of amyloid fibrils reduces the number of amyloid fibrils by at least 50%. In other embodiments, the copolymer penetrates the blood-brain barrier. In some other embodiments, the plurality of amyloid fibrils is disassembled in a brain having amyloid β plaques after receiving an effective amount of the multivalent copolymer.

In various other embodiments, the disclosed multivalent copolymer can reach and disassemble beta amyloid that can be present in the cerebral spinal fluid. The disclosed said copolymer can circulate in the blood and vasculature to reach some specific or all tissues in a living organism. The disclosed multivalent copolymer can disassemble amyloid beta that causes fibrilization of IAPP, which is the protein in the pancreas associated with causing type 2 diabetes. The disclosed multivalent copolymer can disassemble amyloid beta that causes fibrilization for Parkinson's disease in the brain through nucleation of alpha synuclein. The disclosed multivalent copolymer can prevent aggregation of beta amyloid with mPPC, as well as preventing aggregation of amyloid beta and tau protein for Alzheimer's Disease.

The disclosure additionally provides a method of inhibiting the proliferation of amyloid fibrils in a subject at risk of developing amyloid β plaques comprising administering to the subject a multivalent copolymer disclosed herein, wherein the copolymer binds to an amyloid oligomer in the subject, thereby inhibiting a proliferation of amyloid fibrils and formation of amyloid β plaques. In other embodiments, the multivalent copolymer comprises a diagnostic probe for the detection of an amyloid oligomer.

Some aspects this disclosure provide a method of disassembling amyloid fibrils, the method comprising contacting assembled amyloid fibrils with the composition described herein and disassembling the amyloid fibrils.

Some other aspects this disclosure provide a method of disassembling amyloid β plaques in a subject having or suspected of having amyloid β fibrils, the method comprising administering to a subject a therapeutically effective dose of a composition described herein.

Additional aspects of this disclosure provide a method of treating Alzheimer's disease in a subject, the method comprising administering to a subject having or suspected of having Alzheimer's disease a therapeutically effective dose of a composition described herein.

Other aspects of this disclosure provide a method of reversing the symptoms of Alzheimer's disease in a subject, the method comprising administering to a subject having or suspected of having Alzheimer's disease a therapeutically effective dose of a composition disclosed herein.

This disclosure provides ranges, limits, and deviations to variables such as volume, mass, percentages, ratios, etc. It is understood by an ordinary person skilled in the art that a range, such as “numberl” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

### Results and Discussion

The present disclosure provides multivalent polymer-peptide conjugates (mPPCs) as a new class of amyloid fibril breakers that disassemble preformed Aβ fibrils. The kinetics of fibril disappearance is controlled by the molecular weight of the polymer backbone. Atomic force microscopy (AFM) and dynamic light scattering (DLS) studies show that mPPCs effectively transform microscale amyloid fibrils above 400 nm in length into nanostructures under 100 nm in diameter. Circular dichroism (CD) studies and Thioflavin T (ThT) fluorescence assays show that the nanostructures preserve a β-sheet structure, indicating that the disassembly occurs by a direct interaction between mPPCs and intact β-structured Aβ fibrils.

Five mP-iAβ₅ conjugates with the same 7% peptide loading and having a range of molecular weights (22 kDa-224 kDa) were synthesized to investigate the disassembly effect on preformed Aβ₄₀ fibrils. The molecular weight and number of iAβ₅ peptide per polymer chain are summarized in Table 2. We use the notation mP-iAβ₅-22, mP-iAβ₅-46, mP-iAβ₅-90, mP-iAβ₅-166, and mP-iAβ₅-224 to designate the molecular weight of these five mPPCs. Although Aβ₄₂ is more pathogenic species, we chose Aβ₄₀ because Aβ₄₀ is several-fold more than Aβ₄₂ in brain.

The present disclosure demonstrates that synthetic multivalent polymer-peptide conjugates, as the only polymeric amyloid fibrils breaker to date, effectively disassemble preformed Aβ₄₀ fibrils. The molecular weight of mPPCs is a key parameter that determines the rate and extent of fibril disappearance. We envision that the concept described herein could be generalized by changing the peptide moieties on mPPC that specifically interact with different amyloid proteins and disassemble amyloid fibrils in a predictable fashion. The therapeutic potency and the physico-chemical properties of mPPC are currently under investigation.

In one aspect, the present disclosure provides a composition comprising a multivalent polymer backbone conjugated to a plurality of protein binding peptides, wherein the mole % of the protein binding peptides is selected from about 3% to about 12% and the composition having a molecular weight greater than 49 kDa. In one embodiment the molecular weight is selected from about 49 kDa to about 224 kDa. In another embodiment the peptide is an amyloid β (Aβ) binding peptide. In another embodiment, the amyloid β binding peptide has the sequence LPFFD (SEQ ID NO:1).

In another aspect, a copolymer is provided, the copolymer having the structure of Formula A:

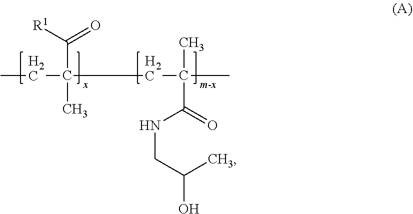

wherein, R₁ is selected from OH and a Aβ binding peptide, and is attached to the repeating polymer structure periodically, having a mole % of the peptide is selected from about 3% to about 12%, wherein x is greater than 8, m is greater than 116, and the molecular weight is greater than about 49 kDa. In one embodiment, x is selected from about 8 to about 61. In another embodiment m is selected from 116 to 1161. In another embodiment, the molecular weight is between 49 kDa and 224 kDa. Formula A is a random copolymer, as disclosed herein. A general synthesis for the preparation of copolymers of Formula I is shown in Scheme A.

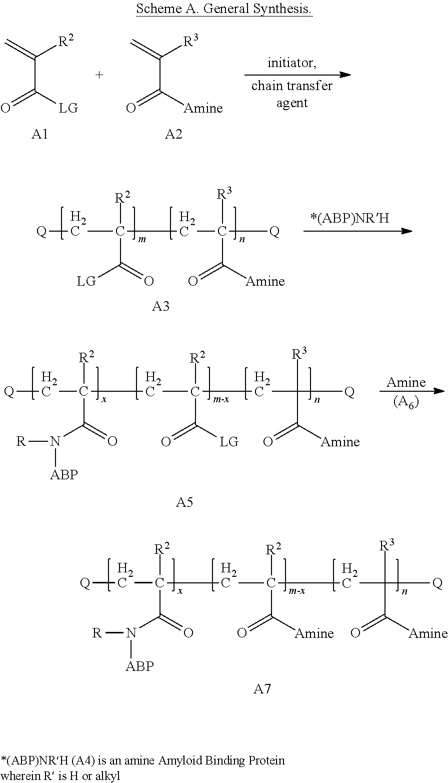

Polymerization of monomers A1 and A2, such as reversible addition-fragmentation chain transfer (RAFT) polymerization using a chain transfer agent, affords copolymer A3, wherein LG is a leaving group that can be partially replaced with amine peptide (A4) to afford copolymer A5. Copolymer A5 can subsequently be treated with amine (A6) to afford copolymer A7, wherein the amine of each polymer segment of A7 can independently be the same or different, and Q is the initiator end and quenched terminal end of the copolymer wherein Q is 2-cyanoprop-2-yl radical, for example, as shown in Scheme B.

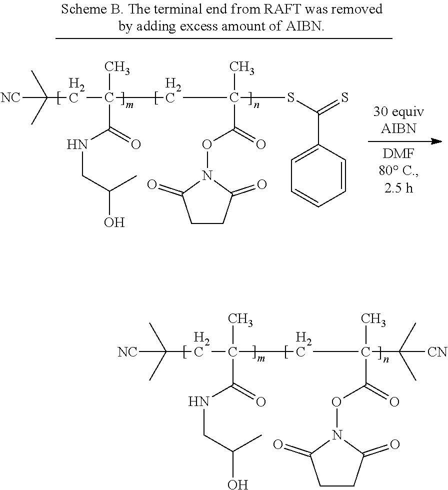 [SMILES: CC(O)CNC(=O)C(C)(CC(C)(C)C#N)CC(C)(SC(=S)c1ccccc1)C(=O)ON1C(=O)CCC1=O ; CC(O)CNC(=O)C(C)(CC(C)(C)C#N)CC(C)(C(=O)ON1C(=O)CCC1=O)C(C)(C)C#N]

In an additional embodiment the Aβ binding peptide has the amino acid sequence LPFFD (SEQ ID NO:1). Additional Aβ binding peptides include, but is not limited to, LVFFA (SEQ ID NO:2), KLVFFA (SEQ ID NO:3), KLVFFAE (SEQ ID NO:4), AIIGL (SEQ ID NO:5), and AH(Met)GL(SEQ ID NO:6).

The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

As used herein, “treat,” “treating” and the like means a slowing, stopping or reversing of progression of a disease or disorder characterized by accumulation of amyloid fibrils (e.g. amyloid β fibrils) when provided a composition described herein to an appropriate control subject. The term also means a reversing of the progression of such a disease or disorder to a point of eliminating or greatly reducing the presence of amyloid fibrils. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

As used herein, “subject” or “patient” means an individual having symptoms of, or at risk for, amyloid fibril accumulation or plaques (e.g. Alzheimer's disease) or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

The compositions described herein may be administered with additional compositions to prolong stability and activity of the compositions, or in combination with other therapeutic drugs.

The compositions described herein could also be used as molecular probes for the detection of amyloid fibrils. Further components of the probes may be: colorants, fluorescent dyes, radioactive isotopes (for example PET), gadolinium (MRI) and/or components which are employed for probes in imaging. After crossing the blood-brain-barrier, the probes may bind to Aβ oligomers and/or plaques. The Aβ oligomers and/or plaques labeled thus can be visualized by means of imaging techniques such as, for example, SPECT, PET, CT, MRT, proton MR spectroscopy and the like.

Amyloid fibril formation is a biological phenomenon that is not unique to Alzheimer's Disease. The protein asynuclein form fibrils that leads to Parkinson's disease. Islet amyloid polypeptide (IAPP) aggregation to fibrils has been linked to type 2 diabetes. Prion proteins that cause Jakob-Creutzfeld Disease in humans, scrapies in sheep, and bovine spongiform encephalopathy (Mad Cow disease). All of these diseases are the result of random coil proteins/peptides that refold in beta-sheet protein structures that aggregate through protein stacking. The aggregates progress to formation of more extensive protein structures known as fibrils. These examples including Aβ proteins are a class of protein known as disordered proteins.

The findings for mPPC polymer class of synthetic molecules may have broader application to other biologically significant disordered proteins that cause other human disease. mPPC polymers with peptide sequences designed to bind to other disordered proteins may similarly inhibit aggregation of these other proteins and may disassemble the corresponding protein fibrils.

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. Nos. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds or copolymers described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound or copolymer is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound or copolymer can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective anti-Alzheimer's agents and have higher potency and/or reduced toxicity as compared to lower molecular weight multivalent copolymers. Preferably, compounds of the invention are more potent and less toxic than multivalent copolymers under 50 kDa, and/or have a different metabolic profile than multivalent copolymers under 50 kDa.

The invention provides therapeutic methods of treating Alzheimer's in a mammal, which involve administering to a mammal having Alzheimer's an effective amount of a compound or composition described herein. A mammal includes a primate, human, rodent, canine, feline, bovine, ovine, equine, swine, caprine, bovine and the like.

The ability of a compound of the invention to treat Alzheimer's may be determined by using assays well known to the art. For example, the design of treatment protocols, toxicity evaluation, data analysis, and quantification of Alzheimer's.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Ability of mp-iAβ₅ Conjugates to Disassemble Amyloid Fibrils

To evaluate the ability of mP-iAβ₅ conjugates to disassemble amyloid fibrils, we conducted experiments in which mP-iAβ₅ conjugates were incubated with preformed Aβ₄₀ fibrils. Freshly prepared Aβ₄₀ (15 μM) solutions were preincubated at 37° C. for 24 h, which is sufficiently long for mature fibrils to grow, as evidenced by AFM images and ThT fluorescence assays (FIG. 8). The preformed Aβ₄₀ fibrils solution was then coincubated with 1.0 equiv of mP-iAβ₅ conjugates of different molecular weights, and the morphology transformation of the fibril structures was monitored at 37° C. for 3 days by DLS, AFM imaging, and ThT fluorescence (we define an equiv of mP-iAβ₅ as the molar ratio of polymer to Aβ₄₀).

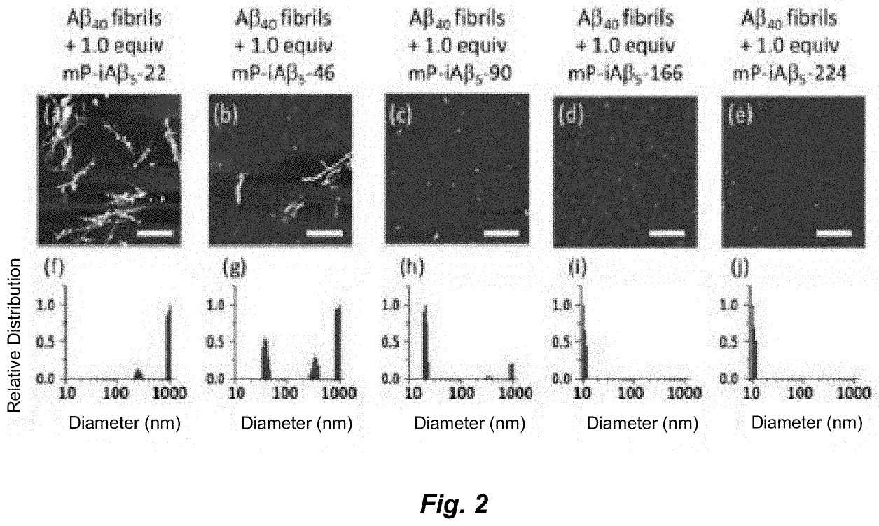

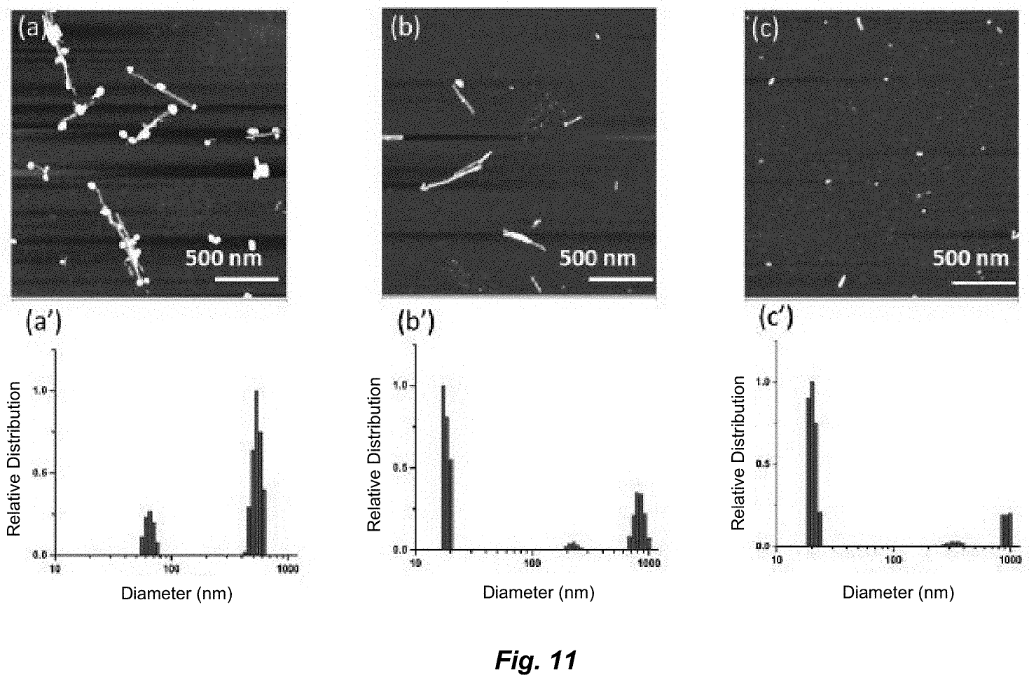

Characterization by AFM and DLS show that as the molecular weight of mP-iAβ₅ conjugates increases, the disassembly effect on Aβ₄₀ fibrils is enhanced and the fraction of nanostructures under 100 nm in diameter increases (FIG. 2). The time-dependent disassembly studies by AFM and DLS over 3 days were summarized in FIG. 11. The molecular weight of mP-iAβ₅ conjugate that completely disassembles preformed Aβ₄₀ fibrils is 166 kDa. AFM images show that mP-iAβ₅-166 efficiently disassembled Aβ₄₀ fibrils, and no fibrils were observed after 2 days (FIG. 2d). The disassembly of preformed fibrils by mP-iAβ₅ conjugates was also quantitatively analyzed by DLS in solution phase. In agreement with AFM images, DLS results confirmed that all Aβ₄₀ fibrils were transformed into dispersible sub-100 nm structures, and 0% of fibrils remained after 3 days of incubation (FIG. 2i). As a control, in the presence of 60.7 equiv of iAβ₅ per Aβ₄₀ (60.7 equiv of iAβ₅ is approximately equal to the concentration of iAβ₅ moieties on 1.0 equiv of mP-iAβ₅-166), the preformed fibrils remained unchanged during 3 days of incubation according to AFM and DLS observations. To investigate the effect of the PHPMA polymer backbone without iAβ₅ moieties on Aβ₄₀ disassembly, we incubated preformed Aβ₄₀ fibrils in the presence of 1.0 equiv of 166 kDa PHPMA with and without 60.7 equiv of iAβ₅ for 3 days. Controls based on PHPMA and the mixtures of PHPMA with iAβ₅ do not have the ability to disassemble the preformed Aβ₄₀ fibrils.

To quantitatively characterize the rate of disappearance of Aβ₄₀ fibrils, DLS was used to monitor the percentage of remaining fibrils above 400 nm and formation of nanostructures under 100 nm over 3 days (Table 1). When Aβ₄₀ fibrils were coincubated with mP-iAβ₅-22, the percentage of fibrils above 400 nm only decreased by 15% after 3 days, and no dispersible sub-100 nm structures were observed. The corresponding AFM images also confirmed the existence of dense fibrils, although the lengths of fibrils are much shorter when compared to the Aβ₄₀ control (FIG. 2a). This finding indicates that, in the presence of low molecular weight mP-iAβ₅ conjugate, the fibril disassembly process is slow and only a fraction of Aβ₄₀ peptides transform from the fibrils to dispersible sub-100 nm structures. As the molecular weight of mP-iAβ₅ conjugates increases, the rate of disappearance of Aβ₄₀ fibrils accelerates and the fraction of sub-100 nm structures increases. The above results demonstrated that mP-iAβ₅ conjugates at the same polymer concentration promote Aβ₄₀ fibril disassembly more efficiently as the molecular weight increases.

TABLE 1
Aβ₄₀ fibril disassembly by mP-iAβ₅ conjugates of different molecular weights monitored by DLS over
3 days. Percentage of structures above 400 nm and under 100 nm are based on DLS histograms.

| | | Equiv of | Day 1 | | Day 2 | | Day 3 | |
|---|---|---|---|---|---|---|---|---|
| Molecular | Equiv of | iAβ₅ | >400 | <100 | >400 | <100 | >400 | <100 |
| Weight | Polymer | Moieties | nm | nm | nm | nm | nm | nm |
| 22 kDa | 1.0 | 8.1 | 91% | 0% | 87% | 0% | 85% | 0% |
| 46 kDa | 1.0 | 16.7 | 84% | 0% | 74% | 26% | 54% | 30% |
| 90 kDa | 1.0 | 32.5 | 79% | 21% | 25% | 71% | 17% | 81% |
| 166 kDa | 1.0 | 60.7 | 12% | 87% | 5% | 94% | 0% | 100% |
| 224 kDa | 1.0 | 81.3 | 8% | 92% | 2% | 97% | 0% | 100% |
| 22 kDa | 2.0 | 16.7 | 92% | 0% | 68% | 30% | 59% | 29% |
| 46 kDa | 1.0 | 16.7 | 84% | 0% | 74% | 26% | 54% | 30% |
| 90 kDa | 0.5 | 16.7 | 81% | 14% | 49% | 42% | 41% | 55% |
| 166 kDa | 0.27 | 16.7 | 65% | 1% | 45% | 55% | 18% | 82% |
| 224 kDa | 0.2 | 16.7 | 69% | 4% | 30% | 61% | 6% | 94% |

To decouple the influence of mP-iAβ₅ molecular weight from the total iAβ₅ moiety concentration, we compared the disassembly effects by keeping the mol concentration of iAβ₅ moieties constant. We define mP-iAβ₅-46 as the standard and reference the concentration of other mP-iAβ₅ conjugates according to their different molecular weights. The mol concentration of corresponding iAβ₅ moieties was thus held constant at 16.7 equiv for all mP-iAβ₅ conjugates.

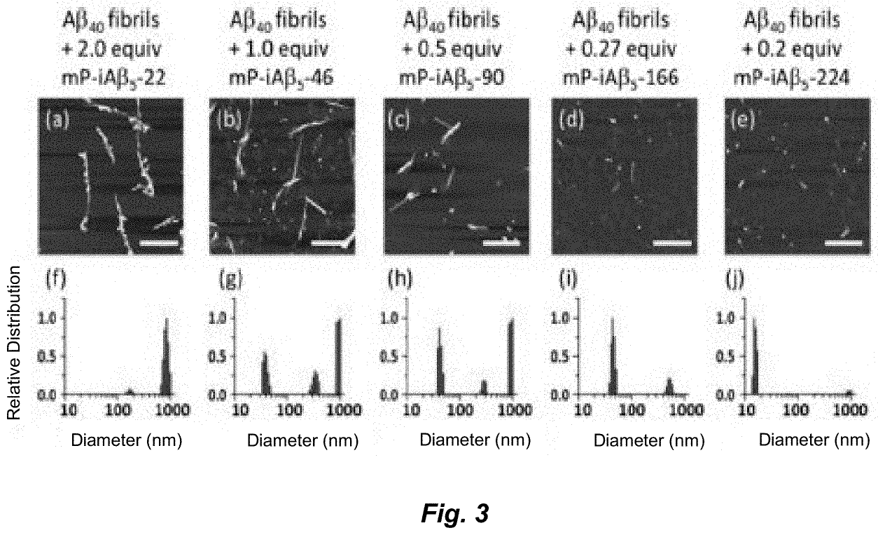

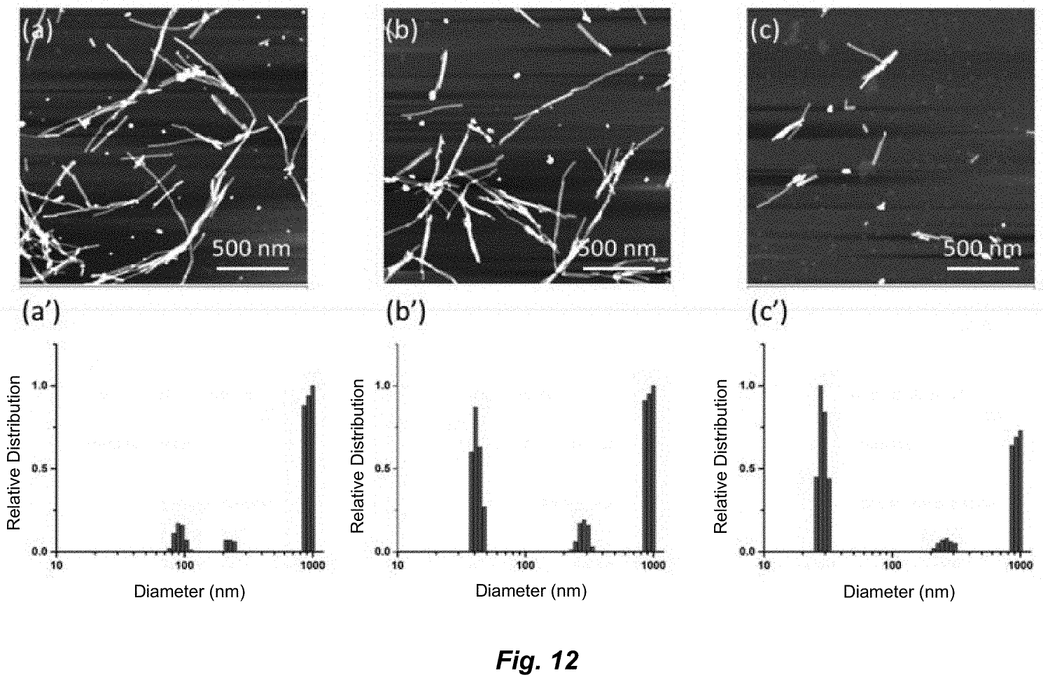

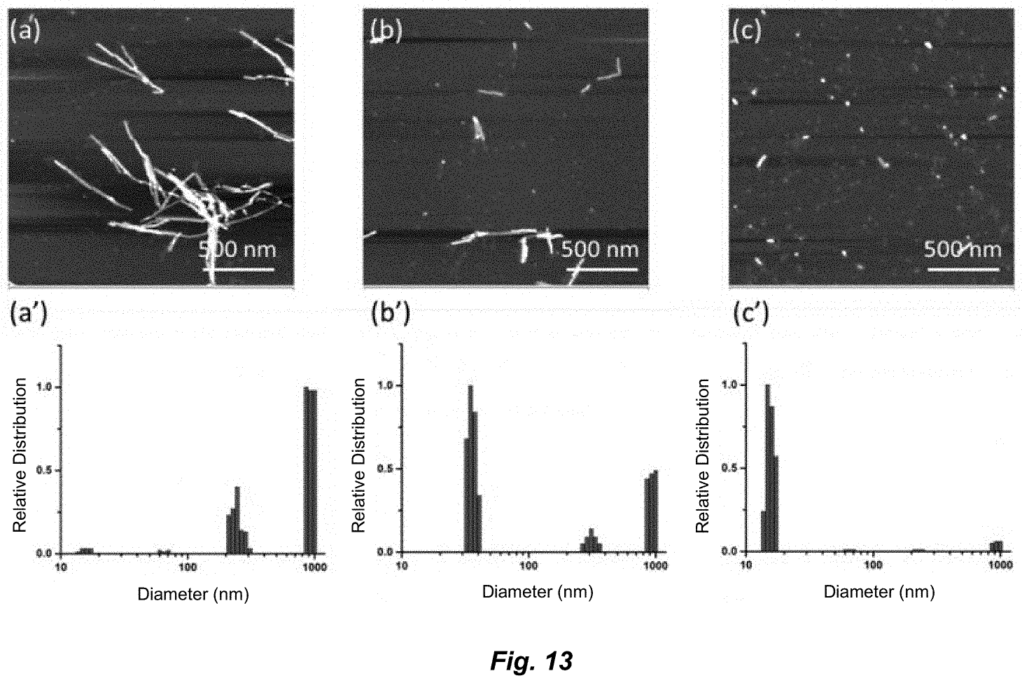

AFM and DLS results demonstrated that as the molecular weight of mP-iAβ₅ conjugates increases, the fraction of remaining fibrils decreases and the fraction of dispersible sub-100 nm structures increases after 3 days (FIG. 3). In addition, the rate of disappearance of Aβ₄₀ fibrils accelerates as the molecular weight of mP-iAβ₅ conjugates increases (Table 1). The time-dependent disassembly studies by AFM and DLS over 3 days were summarized in FIG. 12-13. The mP-iAβ₅-224 disassembles 94% of preformed fibrils after 3 days as indicated by DLS (Table 1), and AFM image shows no fibrillar morphologies (FIG. 3e). These findings demonstrate that mP-iAβ₅ conjugates of higher molecular weights have better disassembly effects on Aβ₄₀ fibrils even when the mol concentration of iAβ₅ moieties is held constant.

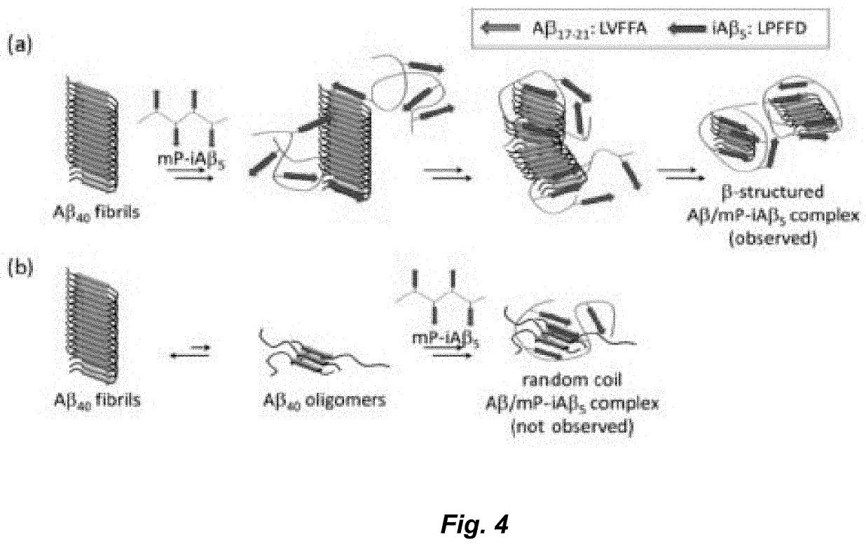

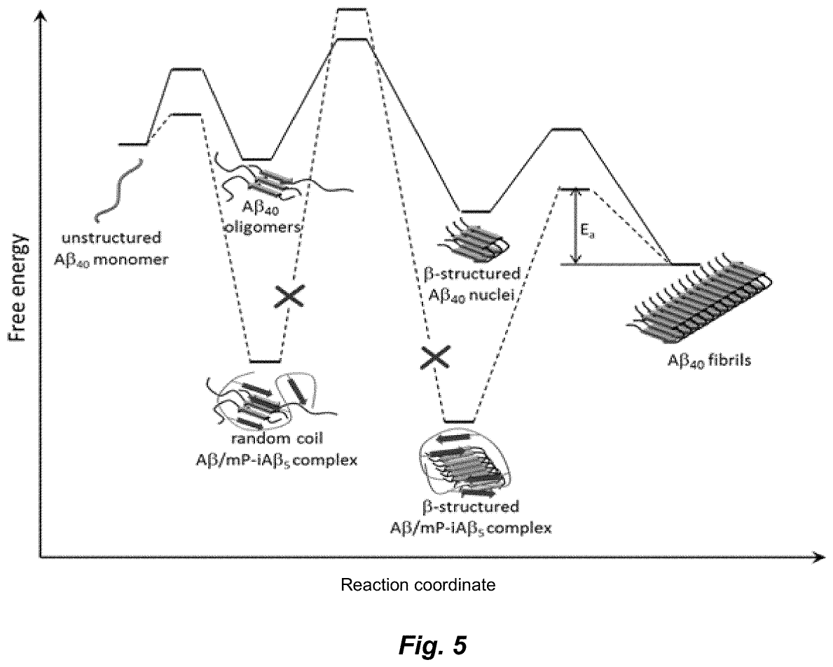

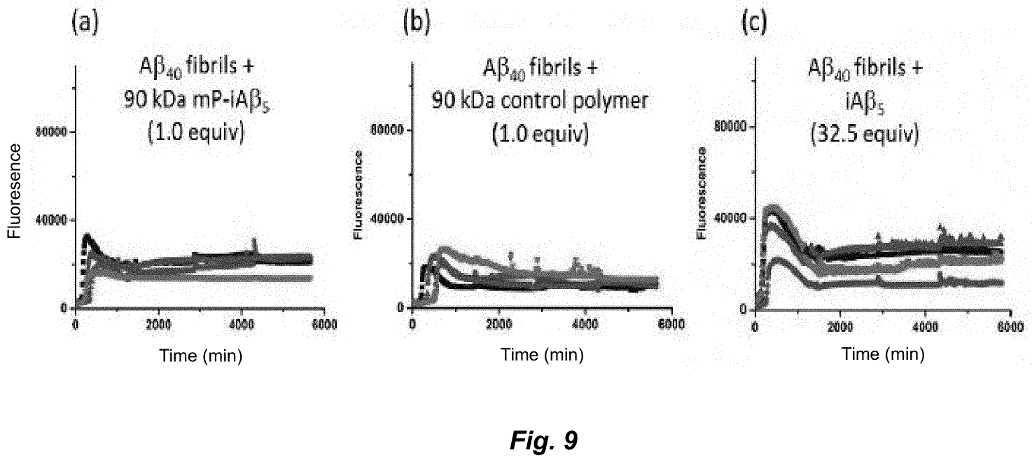

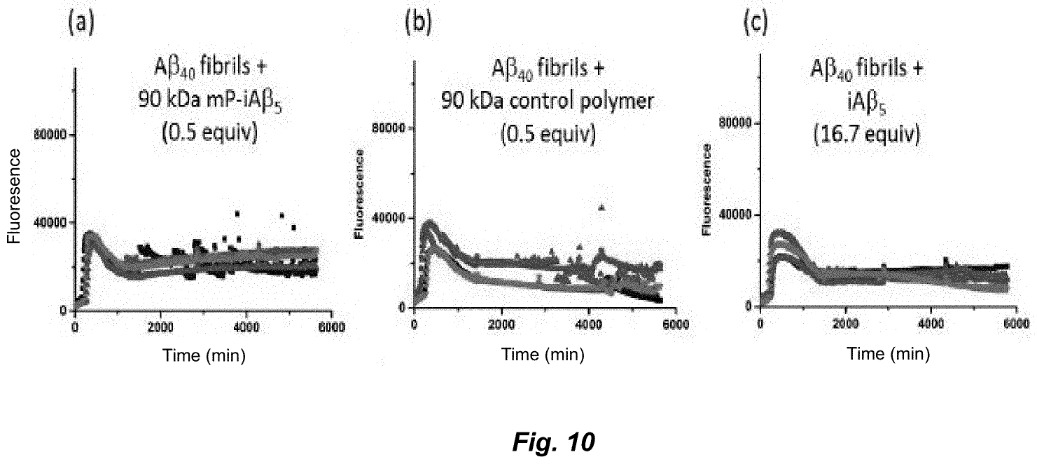

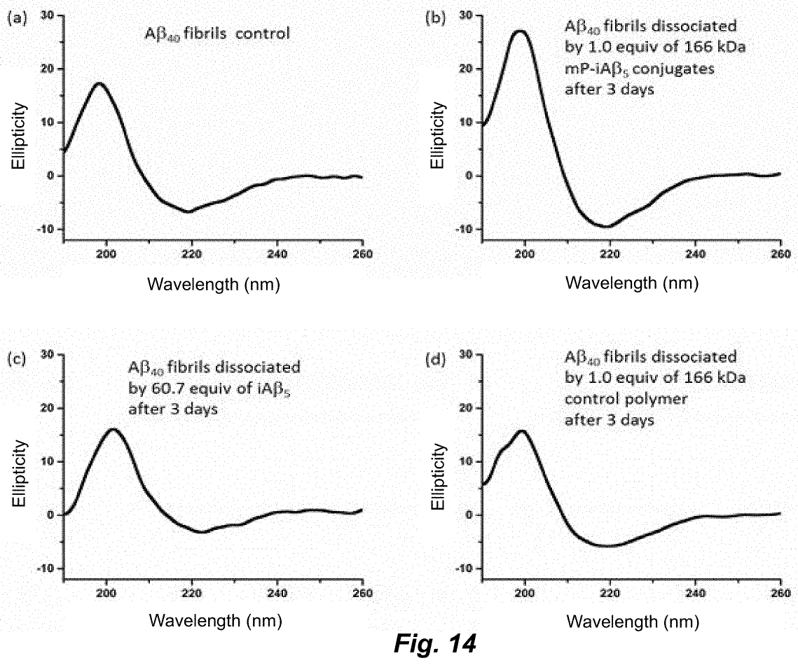

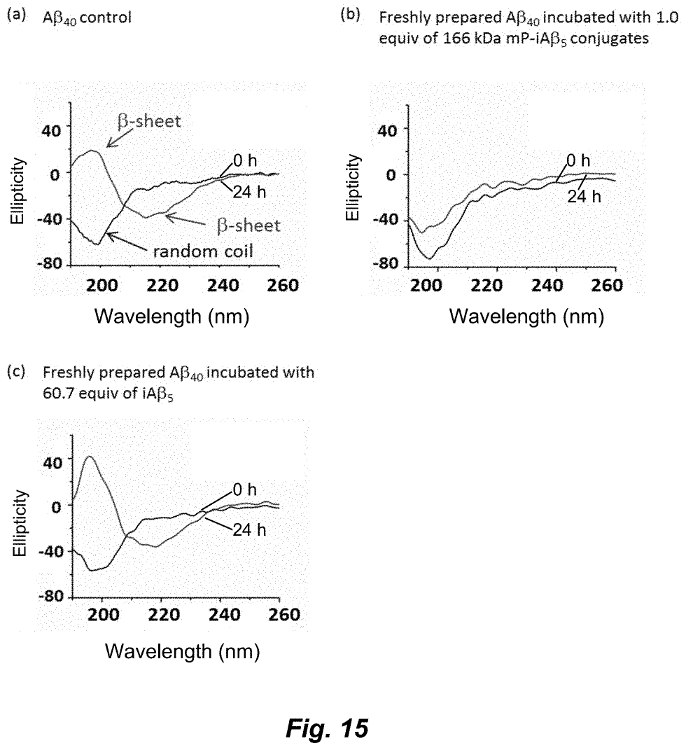

Our studies demonstrate that mP-iAβ₅ conjugates effectively transform preformed Aβ₄₀ fibrils into sub-100 nm structures, and mP-iAβ₅ conjugates promote Aβ₄₀ fibril disassembly more efficiently as the molecular weight increases (Table 1). We propose two hypotheses on disassembly mechanisms (FIG. 4). The first disassembly mechanism is that mP-iAβ₅ conjugates interact with exposed Aβ₄₀ peptides on fibrils (e.g. at the ends of fibrils and defect sites) through multiple specific β-sheet interactions between iAβ₅ and LVFFA sequences. The mP-iAβ₅ conjugates of higher molecular weight create a higher local concentration of iAβ₅ through the multivalent effect. These iAβ₅ moieties complex with Aβ₄₀ and dissemble Aβ₄₀ fibrils at a rate that depends on the effective molarity of iAβ₅ (FIG. 4a). The second disassembly mechanism is that mP-iAβ₅ conjugates bind to Aβ₄₀ monomer and/or oligomers thus shifting the equilibrium between monomeric/oligomeric Aβ₄₀ peptide and Aβ₄₀ fibrils (FIG. 4b). Should equilibration occur, random coil Aβ₄₀ structures are expected since freshly prepared Aβ₄₀ solution with mP-iAβ₅ conjugates result in random coil Aβ₄₀/mP4Aβ₅ complexes (FIG. 5, FIG. 15). We find that Aβ₄₀ fibril disassembly is not accompanied by a decrease in ThT intensity (FIG. 9-10 the variation of ThT intensity may result from Aβ₄₀ fibril morphology), which indicates that the fibril-derived Aβ₄₀ when complexed to mP-iAβ₅ preserves a β-sheet structure. The β-sheet structure of Aβ₄₀/mP-iAβ₅ complex was also confirmed by circular dichroism (FIG. 14). Thus, mP-iAβ₅ conjugates disassemble Aβ₄₀ fibrils by interacting with intact β-structured Aβ₄₀ fibrils rather than monomer and/or oligomers. We do not have direct evidence on the binding between mP-iAβ₅ conjugates and Aβ₄₀ fibrils, but AFM images show nanostructures on the surface of Aβ₄₀ fibrils, which may indicate the interaction between mP-iAβ₅ conjugates and Aβ₄₀ fibrils (FIG. 11a). These results also indicate that Aβ₄₀/mP4Aβ₅ complex generated from Aβ₄₀ fibril disassembly pathway does not interconvert with Aβ₄₀/mP-iAβ₅ complex derived from Aβ₄₀ monomer/oligomers in the inhibition pathway, presumably due to a high energy barrier (FIG. 5). Although the β-structured Aβ₄₀/mP4Aβ₅ complex is thermodynamically stable, the seeding competency remains to be investigated by adding freshly prepared Aβ₄₀ to the solution of disassembled Aβ₄₀/mP4Aβ₅ complex.

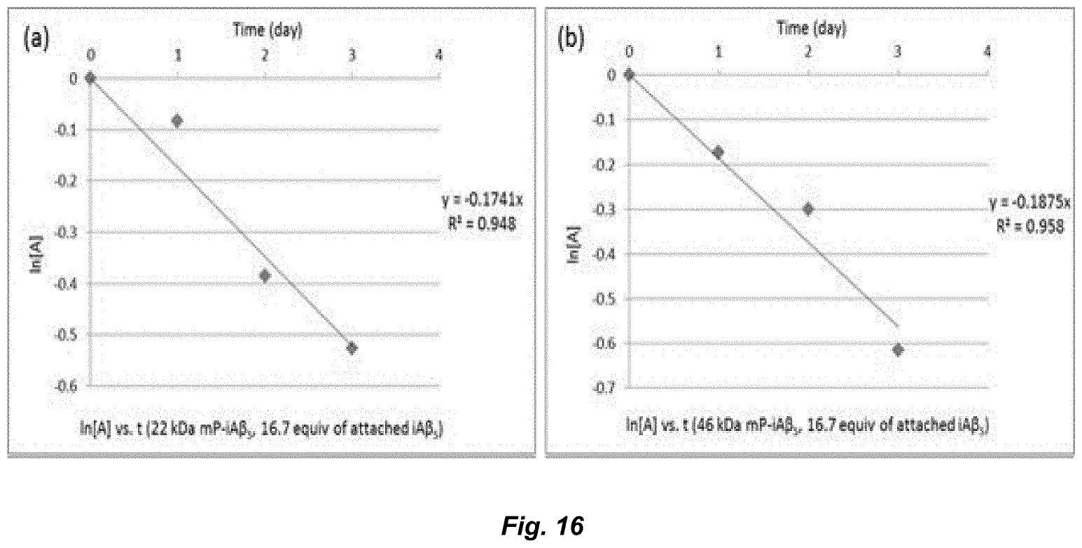

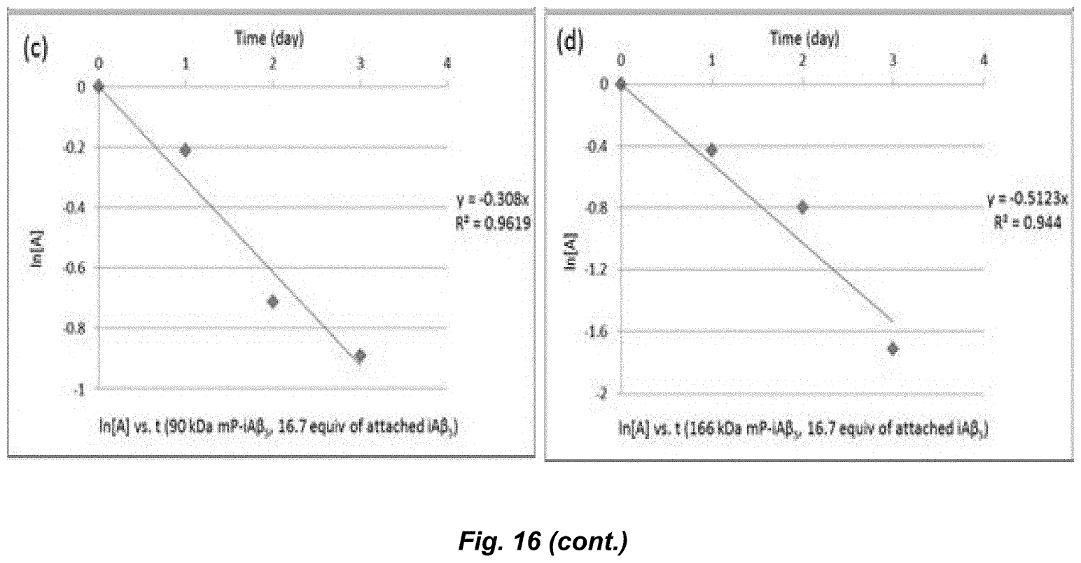

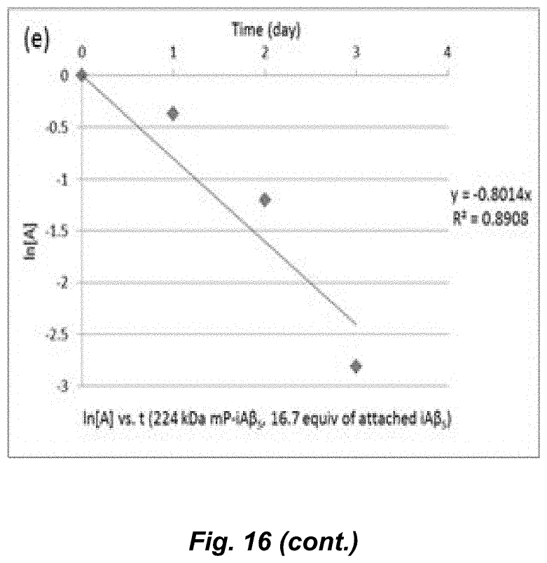

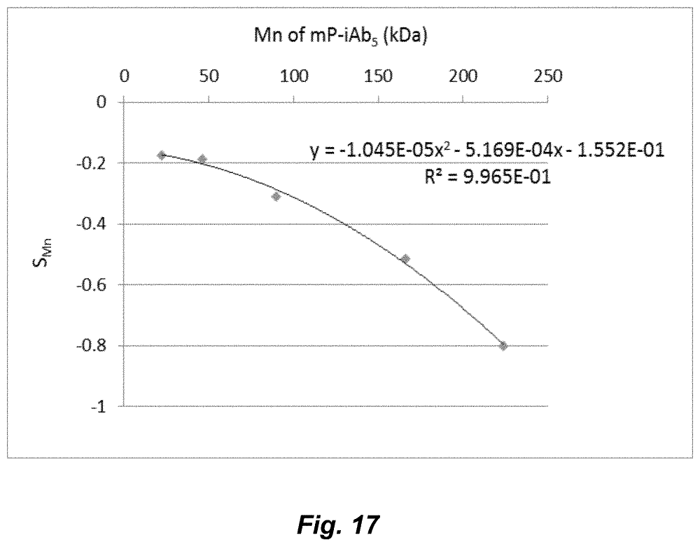

Rate equations for Aβ₄₀ fibrils disappearance are modeled as the percentage of Aβ₄₀ peptide in fibrils above 400 nm vs. time (FIG. 16, 18). Experimentally, the percentage of Aβ₄₀ fibrils and sub-100 nm structures are monitored by DLS (Table 1). Aβ₄₀ monomer is invisible by DLS due to its small size, but it does not affect the percentage of other populations, because the percentage of Aβ₄₀ monomer is negligible based on the reaction coordinate (FIG. 5). The linear regressions of natural log of the fibril percentage vs. time demonstrate that the disassembly of Aβ₄₀ fibrils is a first order reaction in the concentration of Aβ₄₀ fibrils and a pseudo-first order reaction in the concentration of iAβ₅ β-sheet breaker peptide, respectively. We plotted the rate constant k vs. molecular weight and prove that the rate constant k has a positive correlation with the molecular weight of mP-iAβ₅ conjugates (FIG. 17). The faster rate of fibril disassembly for mP-iAβ₅ conjugates of higher molecular weight results from a lower activation energy Eₐ (FIG. 5).

### Example 2

### Aβ₄₀ Fibrils Disassembled by mP-iAβ₅ Conjugates of Different Molecular Weights at a Fixed mol Concentration of Polymer Chains

When Aβ₄₀ fibrils were coincubated with 1.0 equiv of 22 kDa mP-iAβ₅ conjugate, the quantitative analysis by DLS showed that the percentage of fibrils above 400 nm only decreased by 9% after 1 day, 13% after 2 days, and 15% after 3 days. No disassembled sub-100 nm nanostructures were observed. The corresponding AFM images also confirmed the still existence of dense fibrils, although the lengths of fibrils are much shortened compared to Aβ₄₀ control. This finding suggests that, in the presence of low molecular weight mP-iAβ₅ conjugate, the fibrils dissolution process is slow and only a small fraction of Aβ₄₀ disassembles from the fibrils. As control, in the presence of 8.1 equiv of iAβ₅ per Aβ₄₀ (8.1 equiv of iAβ₅ approximately equal the concentration of iAβ₅ moieties on 1.0 equiv of 22 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 1.0 equiv of 22 kDa PHPMA with and without 8.1 equiv of iAβ₅ for 3 days. Controls based on polymer and the mixtures of polymer with iAβ₅ do not have the ability to disassemble the preformed Aβ₄₀ fibrils.

When Aβ₄₀ fibrils were co-incubated with 1.0 equiv of 46 kDa mP-iAβ₅ conjugate, the quantitative analysis by DLS showed that the percentage of fibrils above 400 nm decreased by 16% after 1 day, 26% after 2 days, and 46% after 3 days. The disassembled sub-100 nm nanostructures were not observed after 1 day, 26% after 2 days, and 30% after 3 days. The corresponding AFM images also confirmed the existence of fibrils after 1 day and 2 days, although the lengths of fibrils are much shortened compared to Aβ₄₀ control. The fibrils almost disappeared after 3 days. As control, in the presence of 16.7 equiv of iAβ₅ per Aβ₄₀ (16.7 equiv of iAβ₅ approximately equal the concentration of iAβ₅ moieties on 1.0 equiv of 46 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 1.0 equiv of 46 kDa PHPMA with and without 16.7 equiv of iAβ₅ for 3 days. Controls based on polymer and the mixtures of polymer with iAβ₅ do not have the ability to disassemble the preformed Aβ₄₀ fibrils.

When Aβ₄₀ fibrils were co-incubated with 1.0 equiv of 90 kDa mP-iAβ₅ conjugate, the quantitative analysis by DLS showed that the percentage of fibrils above 400 nm remained 79% after 1 day, 25% after 2 days, and only 17% after 3 days. The disassembled sub-100 nm nanostructures were 21% after 1 day, 71% after 2 days, and 81% after 3 days. The corresponding AFM images confirmed that the fibrils decreased dramatically after 2 days, and almost all the fibrils were converted to the spherical nanostructures after 3 days (FIG. 11). As control, in the presence of 32.5 equiv of iAβ₅ per Aβ₄₀ (32.5 equiv of iAβ₅ approximately equal the concentration of iAβ₅ moieties on 1.0 equiv of 90 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 1.0 equiv of 90 kDa PHPMA with and without 32.5 equiv of iAβ₅ for 3 days. Controls based on polymer and the mixtures of polymer with iAβ₅ do not have the ability to disassemble the preformed Aβ₄₀ fibrils.

The molecular weight of mP-iAβ₅ conjugate to completely disassemble preformed Aβ₄₀ fibrils is 166 kDa. AFM images show that 1.0 equiv of 166 kDa mP-iAβ₅ efficiently induced disassembly of Aβ₄₀ fibrils into spherical nanostructures, achieving almost complete disassembly after 2 days. The disassembly of preformed fibrils by mP-iAβ₅ was also quantitatively analyzed by DLS in solution phase. In consistence with AFM images, DLS results confirmed that all Aβ₄₀ fibrils are broken into sub-100 nm nanostructures, and 0% of fibrils remains after 3 days of incubation. As control, in the presence of 60.7 equiv of iAβ₅ per Aβ₄₀ (60.7 equiv of iAβ₅ approximately equal the concentration of iAβ₅ moieties on 1.0 equiv of 166 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 1.0 equiv of 166 kDa PHPMA with and without 60.7 equiv of iAβ₅ for 3 days. Controls based on polymer and the mixtures of polymer with iAβ₅ do not have the ability to disassemble the preformed Aβ₄₀ fibrils.

When Aβ₄₀ fibrils were co-incubated with 1.0 equiv of 224 kDa mP-iAβ₅ conjugate, the quantitative analysis by DLS were similar to that when Aβ₄₀ fibrils were co-incubated with 166 kDa mP-iAβ₅ conjugates. The corresponding AFM images confirmed that almost all the fibrils were converted to the spherical nanostructures after 1 day, which is faster than the fibril disassembly kinetics of the co-incubation of Aβ₄₀ fibrils and 166 kDa mP-iAβ₅ conjugates. As control, in the presence of 81.3 equiv of iAβ₅ per Aβ₄₀ (81.3 equiv of iAβ₅ approximately equal the concentration of iAβ₅ moieties on 1.0 equiv of 224 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 1.0 equiv of 224 kDa PHPMA with and without 81.3 equiv of iAβ₅ for 3 days. AFM and DLS both demonstrated that the fibrils were fully disassembled after 2 days. It is possible that the PHPMA interact with Aβ₄₀ fibrils through non-specific H-bonding, although the PHPMA have no peptide moiety sequence for specific interaction.

Fibrils Disassembled by mP-iAβ₅ Conjugates of Different Molecular Weights at a Fixed Total Concentration of iAβ₅ Moieties

To decouple the influence of mP-iAβ₅ molecular weight from the total iAβ₅ moiety concentration, we compared the disassembly effects by keeping the mol concentration of iAβ₅ moieties constant. We define mP-iAβ₅-46 as the standard and reference the concentration of other mP-iAβ₅ conjugates according to their different molecular weights. The mol concentration of corresponding iAβ₅ moieties was thus held constant at 16.7 equiv for all mPPCs.

When Aβ₄₀ fibrils were coincubated with 2.0 equiv of 22 kDa mP-iAβ₅ conjugate, the quantitative analysis by DLS showed that the disassembly kinetics were much faster than that when Aβ₄₀ fibrils were coincubated with 1.0 equiv of 22 kDa mP-iAβ₅ conjugate. The percentage of fibrils above 400 nm decreased by 8% after 1 day, 32% after 2 days, and 41% after 3 days. The disassembled sub-100 nm nanostructures were not observed after 1 day, 30% after 2 days and 3 days. The corresponding AFM images also confirmed the existence of fibrils after 1 day and 2 days, although the lengths of fibrils are much shortened compared to Aβ₄₀ control. AFM showed a mixture of short fibrils and disassembled spherical nanostructures after 3 days. As control, in the presence of 16.7 equiv of iAβ₅ per Aβ₄₀ (16.7 equiv of iAβ₅ approximately equal the concentration of iAβ₅ moieties on 2.0 equiv of 22 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 2.0 equiv of 22 kDa PHPMA with and without 16.7 equiv of iAβ₅ for 3 days. Controls based on polymer and the mixtures of polymer with iAβ₅ do not have the ability to disassemble the preformed Aβ₄₀ fibrils.

When Aβ₄₀ fibrils were coincubated with 0.5 equiv of 90 kDa mP-iAβ₅ conjugate, the quantitative analysis by DLS showed that the disassembly kinetics were faster than that when Aβ₄₀ fibrils were coincubated with 2.0 equiv of 22 kDa mP-iAβ₅ conjugate. The percentage of fibrils above 400 nm decreased by 19% after 1 day, 51% after 2 days, and 59% after 3 days. The disassembled sub-100 nm nanostructures were 14% after 1 day, 42% after 2 days, and 55% after 3 days. The corresponding AFM showed a mixture of mature fibrils and disassembled spherical nanostructures after 1 day, the length and number of fibrils decreased significantly after 2 days and 3 days (FIG. 12). As control, in the presence of 16.7 equiv of iAβ₅ per Aβ₄₀ (16.7 equiv of iAβ₅ approximately equal the concentration of iAβ₅ moieties on 0.5 equiv of 90 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 0.5 equiv of 90 kDa PHPMA with and without 16.7 equiv of iAβ₅ for 3 days. Controls based on polymer and the mixtures of polymer with iAβ₅ do not have the ability to disassemble the preformed Aβ₄₀ fibrils.

When Aβ₄₀ fibrils were coincubated with 0.27 equiv of 166 kDa mP-iAβ₅ conjugate, the quantitative analysis by DLS showed that the disassembly kinetics were much faster than that when Aβ₄₀ fibrils were coincubated with 0.5 equiv of 90 kDa mP-iAβ₅ conjugate. The percentage of fibrils above 400 nm decreased by 35% after 1 day, 55% after 2 days, and 82% after 3 days. The disassembled sub-100 nm nanostructures were 1% after 1 day, 55% after 2 days, and 82% after 3 days. The corresponding AFM showed the presence of short fibrils after 1 and 2 days, the fibrils mostly disappeared after 3 days. As control, in the presence of 16.7 equiv of iAβ₅ per Aβ₄₀ (16.7 equiv of iAβ₅ approximately equal the concentration of iAβ₅ moieties on 0.5 equiv of 90 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 0.27 equiv of 166 kDa PHPMA with and without 16.7 equiv of iAβ₅ for 3 days. Controls based on polymer and mixtures of polymer with iAβ₅ do not have the ability to disassemble preformed Aβ₄₀ fibrils.

When Aβ₄₀ fibrils were coincubated with 0.2 equiv of 224 kDa mP-iAβ₅ conjugate, the quantitative analysis by DLS showed that the disassembly kinetics were faster than that when Aβ₄₀ fibrils were coincubated with 0.27 equiv of 166 kDa mP-iAβ₅ conjugate. The percentage of fibrils above 400 nm decreased by 31% after 1 day, 70% after 2 days, and 94% after 3 days. The disassembled sub-100 nm nanostructures were 4% after 1 day, 61% after 2 days, and 94% after 3 days. The corresponding AFM showed the presence of short fibrils after 1 day, the fibrils mostly disappeared after 2 and 3 days (FIG. 13). As control, in the presence of 16.7 equiv of iAβ₅ per Aβ₄₀ (16.7 equiv of iAβ₅ approximately equal the concentration of iAβ₅ moieties on 0.5 equiv of 90 kDa mP-iAβ₅), the preformed fibrils remained unchanged during 3 days of incubation according to AFM and DLS results. To investigate the effect of the PHPMA polymer backbone on Aβ₄₀ fibril disassembly, we incubated Aβ₄₀ fibrils in the presence of 0.2 equiv of 224 kDa PHPMA with and without 16.7 equiv of iAβ₅ for 3 days. Controls based on polymer and the mixtures of polymer with iAβ₅ do not have the ability to disassemble the preformed Aβ₄₀ fibrils.

### Circular Dichroism Studies

We used circular dichroism studies to further confirm the secondary structures of disassembled Aβ₄₀ aggregates in the presence of mP-iAβ₅ conjugates, PHPMA, or iAβ₅. Aβ₄₀ solutions were incubated alone for 24 h to form fibrils. Initially, the CD spectrum of Aβ₄₀ (15 μM) aggregation typically displays a curve with a negative peak at 198 nm, which is characteristic of random coils. As Aβ₄₀ continues to aggregate, the negative peak at 198 nm was converted to the positive peak around 194 nm and a negative peak around 217 nm. The change of the CD spectra indicates the conformational conversion of Aβ₄₀ from random coils to β-sheets, and thus suggests the formation of Aβ₄₀ fibrils.

Fibril breakers (mP-iAβ₅ conjugates, PHPMA, or iAβ₅) were added to preformed Aβ₄₀ fibrils after 24 h and coincubated for 3 days before the structure characterization by circular dichroism. The CD studies showed that the positive peak around 194 nm and negative peak around 217 nm stayed unchanged with 1.0 equiv of 166 kDa mP-iAβ₅ conjugates, which demonstrated that the disassembled aggregates preserved β-sheet structures. These results validate the ThT assays that the fluorescence intensity did not significantly decreased in the disassembly studies. As control, Aβ₄₀ remain β-sheet structures in the presence of PHPMA or iAβ₅, which are consisitent with AFM and DLS results showing that PHPMA or iAβ₅ have no disassembly effect on preformed Aβ₄₀ fibrils.

### Example 3

### Kinetics Studies and Rate Equations of Aβ₄₀ Fibril Disappearance

According to the hypothesis on the disassembly of Aβ₄₀ fibrils by mP-iAβ₅ conjugates, the rate equation of fibril disappearance is expressed as:

r=k_{Mn}[A]^{x}[B]^{y} (1);

where [A] is the concentration of Aβ₄₀ fibrils (>400 nm). There is no absolute concentration, so we use the relative concentration from DLS studies;

[B] is the total concentration of iAβ₅ moieties. [B]=number of iAβ₅ copies on each polymer * mol concentration of mP-iAβ₅;

k_{Mn} is the rate constant, which is determined by molecular weight of mP-iAβ₅ conjugates.

When the total concentration of iAβ₅ moieties was kept constant, the mP-iM35 conjugates of higher molecular weight disassemble Aβ₄₀ fibrils with a faster rate, which indicate that mP-iAβ₅ conjugates increase the local concentration of iAβ₅ by multivalent effect. The multivalent effect alters the rate constant km, in a molecular weight dependent manner.

The concentration of iAβ₅ moieties is the same order as the concentration of Aβ₄₀ monomer, which is much higher than the concentration of Aβ₄₀ fibrils. [B₀]>>[A₀], and the concentration change of iAβ₅ is negligible during the reaction. Thus, the rate equation is modified as:

r=k_{Mn}[A]^{x}[B₀]^{y} (2).

The first step is the determination of x, the reaction order of amyloid fibrils. We use the data in Table 2, in which the concentration of iAβ₅ is kept constant, where

r₂₂=k₂₂[B₀]^{y} [A]^{x} (3);

r₄₆=k₄₆[B₀]^{y} [A]^{x} (4);

r₉₀=k₉₀[B₀]^{y} [A]^{x} (5);

r₁₆₆=k₁₆₆[B₀]^{y} [A]^{x} (6);

r₂₂₄=k₂₂₄[B₀]^{y} [A]^{x} (7);

TABLE 2
Aβ₄₀ fibril disassembly by mP-iAβ₅ conjugates of different molecular weights and same total
concentration of iAβ₅ moieties on pre-incubated Aβ₄₀ (15 μM) fibrils monitored by DLS over
3 days. Percentage of structures above 400 nm and under 100 nm are based on DLS histograms.

| | | Equiv. | Day 1 | | Day 2 | | Day 3 | |
|---|---|---|---|---|---|---|---|---|
| Molecular | Equiv. of | of iAβ₅ | >400 | <100 | >400 | <100 | >400 | <100 |
| Weight | Polymer | Moieties | nm | nm | nm | nm | nm | nm |
| 22 kDa | 2.0 | 16.7 | 92% | 0% | 68% | 30% | 59% | 29% |
| 46 kDa | 1.0 | 16.7 | 84% | 0% | 74% | 26% | 54% | 30% |
| 90 kDa | 0.5 | 16.7 | 81% | 14% | 49% | 42% | 41% | 55% |
| 166 kDa | 0.27 | 16.7 | 65% | 1% | 45% | 55% | 18% | 82% |
| 224 kDa | 0.2 | 16.7 | 69% | 4% | 30% | 61% | 6% | 94% |

To determine x, we plot [A]˜t (zero-order reaction), ln[A]˜t (first order reaction), and 1/[A]˜t (second order reaction). The data has the best fit in linear regression when we plot ln[A]˜t (FIG. 16), so x=1.

When x=1, the rate equations are modified as:

r₂₂=k₂₂[B₀]^{y} [A] (8);

r₄₆=k₄₆[B₀]^{y} [A] (9);

r₉₀=k₉₀[B₀]^{y} [A] (10);

r₁₆₆=k₁₆₆[B₀]^{y} [A] (11);

r₂₂₄=k₂₂₄[B₀]^{y} [A] (12);

Thus, the slope from linear regression is

(S_{Mn})=−k_{Mn}[B₀]^{y} (13);

where [B₀] is a constant in the above studies, k_{Mn} is a function of molecular weight.

Therefore, when k_{Mn}=f(Mn), to determine the relation between k_{Mn} and Mn, we plot S_{Mn} vs. Mn.

The rate equations of Table 3 are represented as:

r′₂₂=k₂₂[B_{0,22}]^{y} [A] (14);

r′₄₆=k₄₆[B_{0,46}]^{y} [A] (15);

r′₉₀=k₉₀[B_{0,90}]^{y} [A] (16);

r′₁₆₆=k₁₆₆[B_{0,166}]^{y} [A] (17);

r′₂₂₄=k₂₂₄[B_{0,224}]^{y} [A] (18).

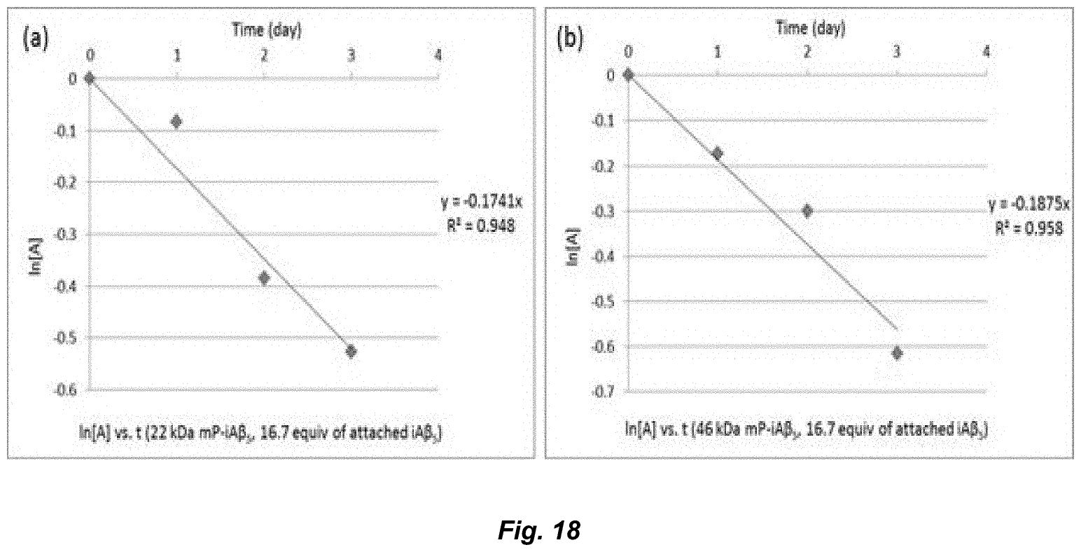

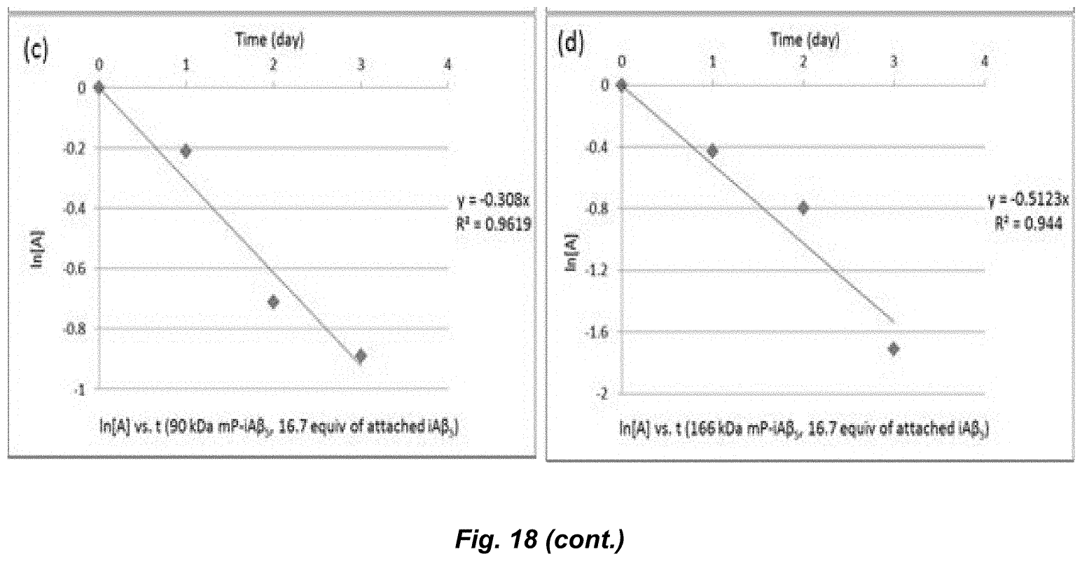

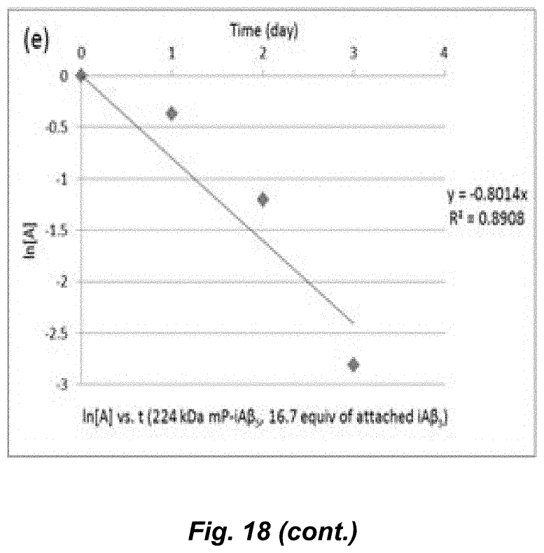

When plotting ln[A]˜t as shown in FIG. 18, the slope is:

(S′_{Mn})=−k₂₂[B_{0,Mn}]^{y} (19).

Thus, [B_{0,Mn}]=0.364 Mn*15 μM, and k_{Mn} is a quadratic function of Mn. From the equation (19) S′_{Mn}=−k_{Mn}[B_{0,Mn}]^{y}, y is determined as 1.

S′_{Mn}˜Mn was also fitted into quadrinomial regression or regression of higher orders, but the value of y is not congruent with the data when comparing the disassembly rate by varying the concentration of iAβ₅ while keeping molecular weight constant. For example, equation (8) to (14), (9) to (15), (10) to (16), etc.

To solve k_{Mn}=f(Mn), we bring the value of y back to equation (13), and S_{Mn}=−k_{Mn}[B₀]^{y} is modified as:

S_{Mn}=−k_{Mn}[B₀] (20);

where [B₀]=16.7*15 μM=250.5 μM; and

S_{Mn}=−k_{Mn}[B₀]=−2.5E−0.4*k_{Mn} (21).

According to the regression equation,

S_{Mn}=−1.045E−05(Mn)²−5.169E−04 (Mn)−0.1552 (22),

the simultaneous equations of (21) and (22) provides:

k_{Mn}=4.18E−02(Mn)²+2.07 Mn+621 (23).

Equation (23) only indicates that k_{Mn} is a quadratic function of Mn. Theoretically, k=Aexp(−Eₐ/RT). The above results suggest that mP-iAβ₅ conjugates of higher molecular weight have lower activation energy Eₐ in the disassembly of Aβ₄₀ fibrils, because their transition state with Aβ₄₀ fibrils has a lower Gibbs energy (FIG. 5). It is also possible that mP-iAβ₅ conjugates of higher molecular weight have bigger pre-exponential factor A, because mP-iAβ₅ conjugates of higher molecular weight create a higher local concentration of properly orientated iAβ₅ moieties, which increase the collision frequency between iAβ₅ moieties and Aβ₄₀ fibrils at a fixed total concentration.

TABLE 3
Aβ₄₀ fibril disassembly by 1.0 equiv of mP-iAβ₅ conjugates of different molecular weights monitored by
DLS over 3 days. Percentage of structures above 400 nm and under 100 nm are based on DLS histograms.

| | | Equiv of | Day 1 | | Day 2 | | Day 3 | |
|---|---|---|---|---|---|---|---|---|
| Molecular | Equiv of | iAβ₅ | >400 | <100 | >400 | <100 | >400 | <100 |
| Weight | Polymer | Moieties | nm | nm | nm | nm | nm | nm |
| 22 kDa | 1.0 | 8.1 | 91% | 0% | 87% | 0% | 85% | 0% |
| 46 kDa | 1.0 | 16.7 | 84% | 0% | 74% | 26% | 54% | 30% |
| 90 kDa | 1.0 | 32.5 | 79% | 21% | 25% | 71% | 17% | 81% |
| 166 kDa | 1.0 | 60.7 | 12% | 87% | 5% | 94% | 0% | 100% |
| 224 kDa | 1.0 | 81.3 | 8% | 92% | 2% | 97% | 0% | 100% |

In this group of study, [B₀]=number of iAβ₅ per chain (n)*mol concentration of mP-iAβ₅ conjugates (m). According to the data shown in Table 4, n=0.364 Mn, m=15 μM, [B_{0, Mn}]=0.364 Mn*15 μM.

TABLE 4
Degree of polymerization and number of iAβ₅ per chain of
mP-iAβ₅ conjugates of different molecular weights.

| | | Loading | Degree of | Number of iAβ₅ |
|---|---|---|---|---|
| Molecular Weight | | Ratio | Polymerization | per Chain |
| 22 | kDa | 7% | 116 | 8.1 |
| 46 | kDa | 7% | 239 | 16.7 |
| 90 | kDa | 7% | 464 | 32.5 |
| 166 | kDa | 7% | 867 | 60.7 |
| 224 | kDa | 7% | 1161 | 81.3 |

In conclusion, the rate equation of fibril disappearance is determined as, r=k_{Mn}[B₀] [A], where k_{Mn} is dependent on Mn in a quadratic manner.

### Example 4

### Materials and Methods

Materials. N-hydroxysuccinimide methacrylate (NHSMA, 98%), 2-cyano-2-propyl benzodithioate (CIDB, >97%), Thioflavin T (ThT, dye content, 75%), dimethyl sulfoxide (DMSO, anhydrous, >99.9%), N, N′-dimethylformamide (DMF, anhydrous, 99.8%), and tert-butanol (anhydrous, 99.5%) were purchased from Sigma-Aldrich and used as received. 2,2′-azobis(2-methylpropionitrile) perphenazine (AIBN, 98%) was purchased from Sigma-Aldrich and recrystallized before use. 1-amino-2-propanol (94%) was purchased from Acros. N-(2-hydroxypropyl) methacrylamide (HPMA, 98%) was purchased from Polyscience. Amyloid protein (Aβ₄₀) was purchased from GL Biochem Ltd. (Shanghai, China). Pentapeptide LPFFD (iAβ₅) was purchased from American Peptide. Molecular biology grade water (ultrapure water) for ThT Assays was purchased from Corning. PBS buffer (100 mM) was purchased from Lonza. All other solvents (HPLC or spectroscopic grade) were purchased from Sigma-Aldrich or Fisher, and used as received.

Nuclear Magnetic Resonance (NMR). ¹H NMR spectra were obtained on a Varian Unity 400 spectrometer in the School of Chemical Sciences NMR laboratory at the University of Illinois at Urbana-Champaign. Spectra were referenced to residual solvent peaks. Chemical shifts are expressed in parts per million (δ). NMR deconvolution was done on the software MestReNova.

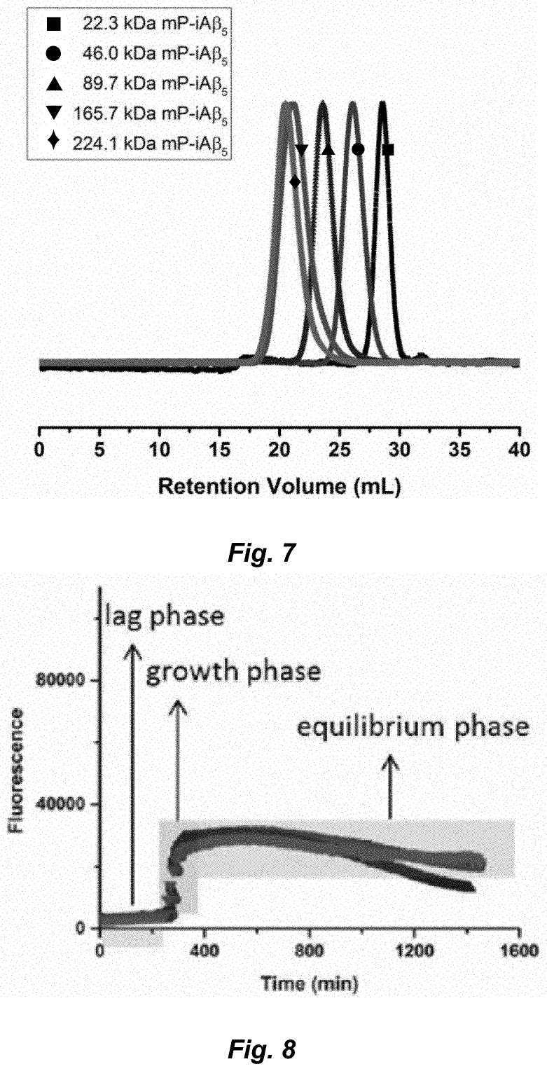

Gel Permeation Chromatography (GPC). The molecular weight and polydispersity were determined by gel permeation chromatography (Breeze 2 GPC, Waters), with Styragel HT column (Waters). Dimethylformamide (DMF) containing 20 mM LiBr was used as the eluent, with the elution rate of 1 mL min⁻¹ (FIG. 7). Polystyrene standards were used for calibration.

Atomic Force Microscopy (AFM). Samples for AFM studies were taken directly from the ThT assays, and 10 μL of each sample solution was loaded on freshly cleaved mica surface (Electron Microscopy Sciences, catalog NO. 71856-01). Samples were incubated for 5 minutes and rinsed with 5 drops of molecular biology grade water. The mica surface was blow-dried under nitrogen. AFM images were obtained on a MultiMode V (Bruker, Santa Barbara, USA) microscope in tapping mode. Ultrasharp silicon cantilevers (SCANASYST-ATR, Bruker) were used. All of the images were collected at a scan rate of 1 Hz and scan lines of 512.

Dynamic Light Scattering (DLS). Samples for DLS studies were prepared at the same condition as in ThT assays without adding ThT dye molecules. The DLS studies were carried out using a particle sizer NICOMP 380 ZLS.

Circular Dichroism (CD) spectroscopy. CD spectra of Aβ₄₀ fibril solution incubated without or with fibril breakers (mP-iAβ₅ conjugates, PHPMA, or iAβ₅) were recorded in a JASCO J-815 Spectrometer (JASCO Co., Tokyo, Japan), using a quartz cuvette (1 mm path length). The concentration of Aβ₄₀ solution for CD analysis was 15 μM and the Aβ₄₀ solutions incubated without or with fibril breakers were prepared at 1.0 equiv (we define equiv as the molar ratio of modulators to Aβ₄₀). The spectra were taken as the average of three accumulations from 190 and 260 nm at a speed of 50 nm/min. All of the samples were incubated at 37° C. in 10 mM phosphate buffer solution (PBS) with a continuous agitation speed of 567 rpm. Spectra were calibrated by subtracting the buffer or sample solution baseline.

Synthesis of Multivalent PHPMA-iAβ₅ (mP-iAβ₅) Conjugates

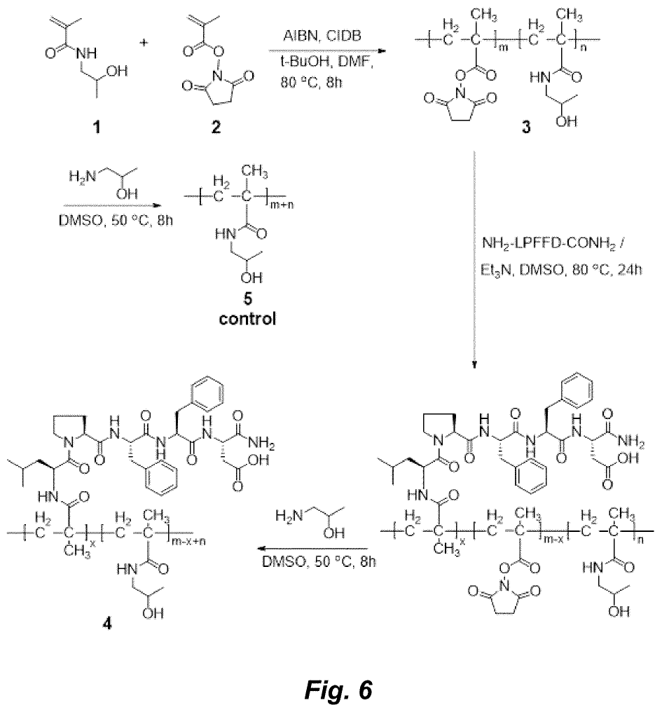

We followed the previous synthetic strategy to synthesize mP-iAβ₅ conjugates and PHPMA (Scheme 1, FIG. 6), and the molecular weight was controlled by tuning the relative ratio between monomer and initiator.

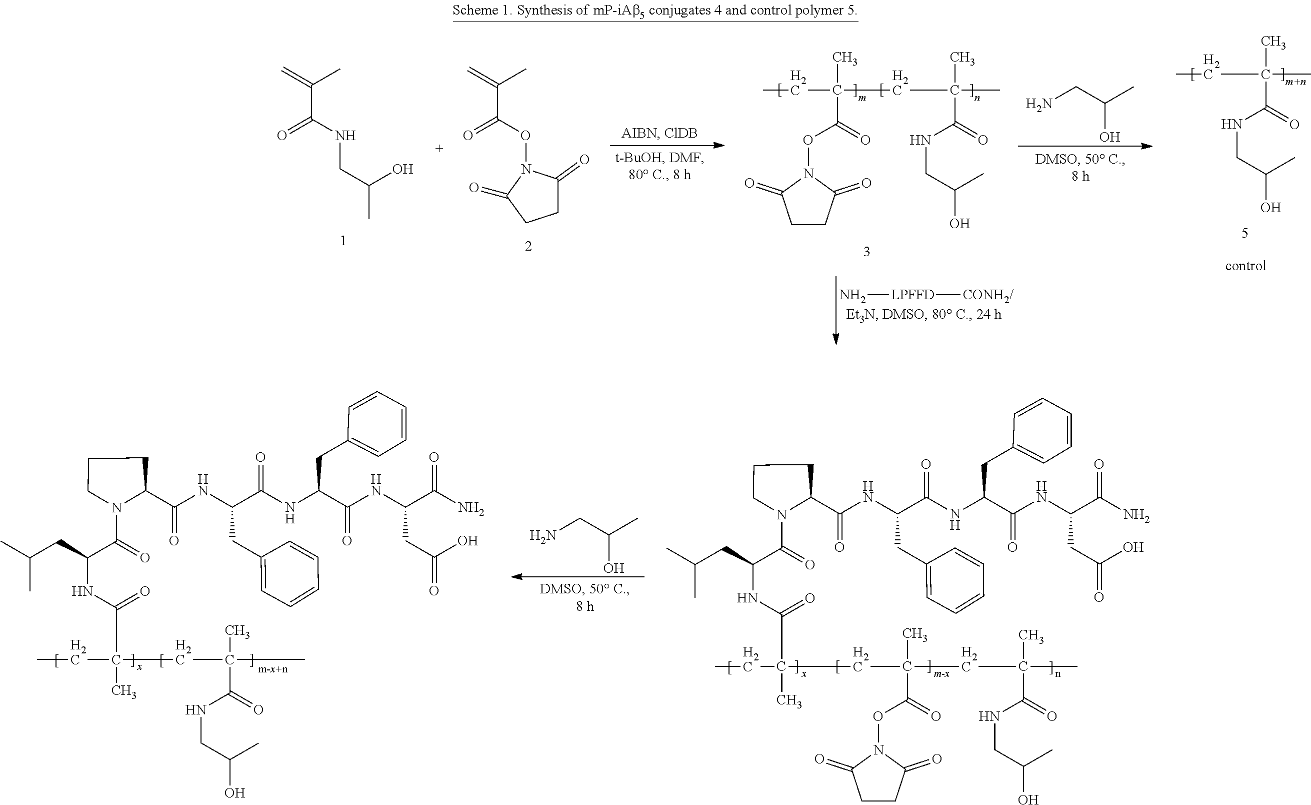

The composition of copolymer 3 was determined by ¹H NMR spectroscopy (mol % of NHSMA and mol % of HPMA). ¹H NMR (400 MHz, DMSO-d₆) δ 7.85˜7.73 (br, Ph-CSS—), 7.73˜7.00 (br, —CO—NH—), 4.78˜4.41 (br, HO—CH(CH₃)—CH₂—), 3.90˜3.47 (br, HO—CH(CH₃)—CH₂—), 3.20-2.55 (br, HO—CH(CH₃)—CH₂—, succinimide), 2.40˜1.47 (br, —CH₂—C—), 1.46-0.42 (br, CH₃—).

The loading ratio of peptides iAβ₅ moieties on mP-iAβ₅ conjugates 4 was determined by ¹H NMR spectroscopy (7% loading ratio, Table 5). ¹H NMR (400 MHz, CD₃OD) δ 7.72˜7.35 (br, —CO—NH— in PHPMA), 7.35-6.90 (br, phenyl groups in iAβ₅), 4.05˜3.73 (br, HO—CH(CH₃)—CH₂—), 3.27˜2.78 (br, d, HO—CH(CH₃)—CH₂—), 2.45-1.56 (br, —CH₂—C— in polymer backbone), 1.55˜0.73 (br, CH₃—). FT-IR (cm⁻¹): 3687-3060 (O—H, N—H), 2974 (—CH₃), 2935 (—CH₂—), 1665 (amide I), 1564 (phenyl on penta-peptide), 1535 (amide II), 1205 (C—O).

Molecular weight and polydispersity were determined by DMF GPC. The degree of polymerization and number of iAβ₅ per chain are calculated accordingly (Table 5).

TABLE 5-1
Molecular weight, PDI, and number of iAβ
₅ of mP-iAβ₅ conjugates 4.

| | | | Loading | Degree of | Number of iAβ₅ |
|---|---|---|---|---|---|
| Molecular Weight | | PDI | Ratio | Polymerization | per Chain |
| 22 | kDa | 1.1 | 7% | 116 | 8.1 |
| 46 | kDa | 1.1 | 7% | 239 | 16.7 |
| 90 | kDa | 1.3 | 7% | 464 | 32.5 |
| 166 | kDa | 1.5 | 7% | 867 | 60.7 |
| 224 | kDa | 1.4 | 7% | 1161 | 81.3 |

Thioflavin T Fluorescence Assays

TABLE 5-2
Deconvolution example of Loading Ratio result of mP- iAβ₅-7% 4
from ¹H NMR spectrum.

| Moiety | Shift (ppm) | Area | ¹H |
|---|---|---|---|
| LPFFD | 7.27 | 5653 | 5 |
| LPFFD | 7.21 | 10642 | 5 |
| HPMA | 3.16 | 25804 | 1 |
| HPMA | 3.00 | 18607 | 1 |

$Loading ratio = \frac{( 5653 + 10642 ) / 10}{\frac{25804 + 18607}{2} + \frac{5653 + 10642}{10}} = 7 \%$

ThT fluorescence assays were conducted in 96-well black plate (Thermo Scientific NUNC, catalog NO. 265301) at 37° C. with continuous shaking (567 rpm) in a BioTek Hybrid H1 plate reader. ThT fluorescence was recorded with 10 minutes reading intervals and 5 s shaking before first read (442 nm excitation, 482 nm emission). All samples were run in quadruple or more. At least three independent experiments were carried out for each ThT assay. Each well contained 10 mM PBS buffer solution (pH 7.4) and 20 μM ThT in a total volume of 200 μL. Aβ₄₀ (15 μM, 1.0 equiv), iAβ₅, control polymer 5, and mP-iAβ₅ conjugates 4 of different molecular weights were added as needed. The concentrations of added samples were calculated based on Aβ₄₀ (15 μM, 1.0 equiv).

Preparation of Buffered ThT Solution. The ThT solution in 20 mM PBS was freshly prepared before use. Thioflavin T (4 mg) was dissolved in 20 mL of ultrapure water and filtered through a 0.22 micron filter. The concentration of the above solution was determined by UV-Vis at 412 nm (ε=36000 M⁻¹ cm⁻¹). Based on the determined concentration of the ThT solution, ultrapure water and 100 mM PBS were further added to dilute the ThT solution to 40 μM in 20 mM PBS, pH 7.4

Preparation of Afizio Solution. The Aβ₄₀ solution was freshly prepared as following. Aβ₄₀ was dissolved in 100 mM NaOH (aq) to the concentration of 1.5 mM and sonicated for 30 seconds. The resulting solution was diluted to 300 μM by adding ultrapure water. The solution was filtered through 100 kDa centricon filters (Pall Life Sciences, catalog NO. OD100C34) at 8000 rpm for 8 minutes to remove any pre-aggregates. The freshly prepared solution was further diluted to 150 μM by ultrapure water and used for assays.

Sample Preparation. Conjugates 4, control polymer 5, and pentapeptide iAβ₅ were dissolved in ultrapure water to 10 times concentrated as needed concentration in ThT assays. The filtered solutions were further diluted to different concentrations in fibril disassembly assays.

Preparation of Solutions for 96-well Plate. The buffered ThT solution, Aβ₄₀ solution (150 μM, 20 μL), and ultrapure water were mixed in a certain ratio and added to each well so that each well contained 20 μM ThT, 10 mM PBS, and 15 μM Aβ₄₀. The above solution was incubated for 24 h to form Aβ₄₀ fibrils, then different concentration of fibril breakers (mP-iAβ₅ conjugates 4, control polymer 5, and iAβ₅) were added to each well to disassemble preformed Aβ₄₀ fibrils. The detailed preparation of 96-well plate is shown as following:

To each well of 96-well plate, the buffered ThT solution (40 μM, 100 μL) and ultrapure water (80 μL) were added and mixed. Then Aβ₄₀ solution (150 μM, 20 μL) was added. The samples of pure Aβ₄₀ were incubated for 24 h to form mature fibrils. Then mP-iAβ₅ conjugates, PHPMA, and iAβ₅ were dissolved in ultrapure water to 10 times concentrated as needed concentration in ThT assays. The above concentrated solutions of modulators (20 μL) were added to the wells containing 200 μL of pre-incubated Aβ₄₀ solutions to reach the needed concentration. The samples containing mature fibrils of Aβ₄₀ and modulators were coincubated for another 3 days in plate reader. The fluorescence was monitored by ThT assays.

ThT Assays of Aβ₄₀ Fibril Disassembly by mP-iAβ₅ Conjugates 4. ThT assays of Aβ₄₀ aggregation typically display a sigmoidal curve comprising three phases: lag phase, growth phase, and equilibrium phase (FIG. 8). The lag phase generally corresponds to lack of mature Aβ₄₀ fibrils. The rapid growth phase indicates increasing Aβ₄₀ fibrils concentration. Finally, the aggregation process reaches equilibrium phase when most of Aβ₄₀ peptides are converted to mature fibrils. Different color data points correspond to data from multiple runs under identical experimental conditions in the same plate.

When Aβ₄₀ fibrils are co-incubated with mP-iAβ₅ conjugates, PHPMA, and iAβ₅, fibril disassembly is not accompanied by the decreased ThT intensity, which indicates that the disassembled Aβ₄₀ still preserve β-sheet structure. The β-sheet structure of disassembled Aβ₄₀ protein was also confirmed by circular dichroism (FIG. 14).

TABLE 6
Summary of modulatory effects of polymer-peptide conjugates
with different peptide sequences on Aβ₄₀ (15 μM).

| Peptide | | | |
|---|---|---|---|
| Sequences on | | | |
| Conjugates (7%) | ThT | AFM | Comments |
| LPFFD-CONH₂ | Lag time of 650 min at 0.1 | Nanostructures at 0.5 | Lead sequence. |
| (SEQ ID NO: 1) | equiv. | and 1.0 equiv. | |
| | No fluorescence response | | |
| | at 0.5 and 1.0 equiv. | | |
| LVFFA-CONH₂ | N/A | N/A | The conjugates are |
| (SEQ ID NO: 2) | | | insoluble. |
| LVFFA-COOH | Flat lines with | Short and rigid fibrils. | LVFFA pentapeptide |
| (SEQ ID NO: 2) | fluorescence response at | | itself aggregates into |
| | 0.5 and 1.0 equiv. | | fibrils. |
| KLVFFA-CONH₂ | N/A | N/A | The conjugates form gel |
| (SEQ ID NO: 3) | | | in buffer solutions. |
| KLVFFAE-CONH₂ | Lag time of 910 min at 0.1 | Short fibrils at 0.1 | Viscous solutions. |
| (SEQ ID NO: 4) | equiv. | equiv. | |
| | Flat lines with | Nanostructures at 0.5 | |
| | fluorescence response at | and 1.0 equiv. | |
| | 0.5 and 1.0 equiv. | | |
| AIIGL-COOH | Lag time of 220 min at | Fibrils | The interaction between |
| (SEQ ID NO: 5) | 0.1, 0.5, and 1.0 equiv, the | | LVFFA and AIIGL is |
| | same as Aβ₄₀ control | | weak. |
| AII(Met)GL-COOH | | | |
| (SEQ ID NO: 6) | | | |

### Example 5

### Pharmaceutical Dosage Forms

The following formulations illustrate representative pharmaceutical dosage forms that may be used for the therapeutic or prophylactic administration of a compound or copolymer of a formula described herein, a compound or copolymer specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to lOOg |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl mono stearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1.-10. (canceled)

11. A method of disassembling an amyloid fibril comprising contacting an amyloid fibril with a multivalent random copolymer comprising Formula I: 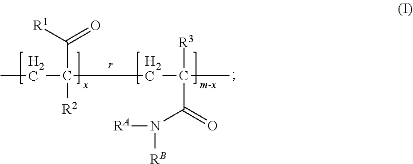
wherein
R¹ is an Amyloid β binding peptide;
R² and R³ are each independently (C₁-C₃)alkyl;
R^{A} is H or methyl;
R^{B} is (C₁-C₆)alkyl or (C₃-C₆)cycloalkyl wherein the alkyl or cycloalkyl is optionally monosubstituted with OH or NH₂;
m is 100 to 2000;
x is 1 to 200; and
the number average molecular weight of the copolymer is about 20 kDa to about 500 kDa;
wherein the r between the x and m-x segments indicates that Formula I is a random copolymer;
wherein the random copolymer binds to the amyloid fibril and at least partially disassembles the secondary structure of the amyloid fibril into one or more nanostructures having a length of less than about 400 nm.

12. The method of claim 11 wherein the nanostructures have a length of less than 100 nm.

13. The method of claim 11 wherein the nanostructures have a diameter of less than 100 nm.

14. (canceled)

15. (canceled)

16. The method of claim 11 wherein the random copolymer penetrates the blood-brain barrier.

17. The method of claim 16 wherein disassembling a plurality of amyloid fibrils reduces the number of amyloid fibrils by at least 50%.

18. The method of claim 17 wherein the plurality of amyloid fibrils is disassembled in a brain having amyloid β plaques after receiving an effective amount of the random copolymer.

19. (canceled)

20. (canceled)

21. The method of claim 11 wherein R¹ is LPFFD (SEQ ID NO:1), LVFFA (SEQ ID NO:2), KLVFFA (SEQ ID NO:3), KLVFFAE (SEQ ID NO:4), AIIGL (SEQ ID NO:5), or AH(Met)GL (SEQ ID NO:6).

22. The method of claim 21 wherein R¹ is LPFFD (SEQ ID NO:1).

23. The method of claim 11 wherein R^{B} is —CH₂CH(OH)CH₃.

24. The method of claim 11 wherein the binding peptide (R¹) has a loading ratio of about 1% to about 25% of the monomer segments of the copolymer.

25. The method of claim 24 wherein the loading ratio is about 5% to about 10%.

26. The method of claim 11 wherein m is about 250 to about 1200.

27. The method of claim 11 wherein x is about 33 to about 81.

28. The method of claim 11 wherein the number average molecular weight of the random copolymer is about 50 kDa to about 500 kDa.

29. The method of claim 28 wherein the number average molecular weight of the random copolymer is about 125 kDa to about 400 kDa.

30. The method of claim 28 wherein the number average molecular weight of the random copolymer is about 125 kDa to about 300 kDa.

31. A method of disassembling an amyloid fibril comprising contacting an amyloid fibril with a multivalent random copolymer comprising Formula I: 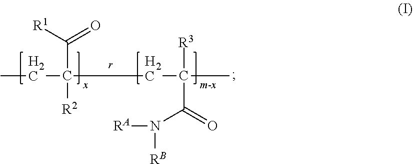
wherein
R¹ is LPFFD (SEQ ID NO:1);
R² and R³ are each independently (C₁-C₃)alkyl;
R^{A} is H or methyl;
R^{B} is —CH₂CH(OH)CH₃;
m is about 464 to about 1161;
x is about 33 to about 81; and
the number average molecular weight of the copolymer is about 90 kDa to about 224 kDa and R¹ has a loading ratio of about 7% of the monomer segments of the random copolymer;
wherein the r between the x and m-x segments indicates that Formula I is a random copolymer;
wherein the random copolymer binds to the amyloid fibril and at least partially disassembles the secondary structure of the amyloid fibril into one or more nanostructures having a length of less than about 400 nm.

32. The method of claim 31 wherein the number average molecular weight of the random copolymer is about 90 kDa and its degree of polymerization is about 464.

33. The method of claim 31 wherein the number average molecular weight of the random copolymer is about 166 kDa and its degree of polymerization is about 867.

34. The method of claim 31 wherein the number average molecular weight of the random copolymer is about 224 kDa and its degree of polymerization is about 1161.
